(19) United States
(12) Patent Application Publication
(10) Pub. No.: US 2019/0098920 A1
(43) Pub. Date: Apr. 4, 2019

(54) CEREAL GRAIN PROCESSING

(71) Applicant: DUPONT NUTRITION BIOSCIENCES APS, Copenhagen K (DK)

(72) Inventor: Eduardo Wilson Santos Pimentel, Sao Paulo (BR)

(21) Appl. No.: 15/554,037

(22) PCT Filed: Mar. 1, 2016
(86) PCT No.: PCT/US2016/020246

§ 371 Date: Aug. 28, 2017

Related U.S. Application Data
(60) Provisional application No. 62/128,066, filed on Mar. 4, 2015.

(51) Int. Cl. A23L 7/104; A23L 7/10; C08B 37/00; C12P 19/14

(52) U.S. Cl. CPC A23L 7/107; A23L 7/197; C12P 19/14; C08B 37/0024; C08B 37/0057; A23L 7/198

(57) ABSTRACT

The present disclosure provides systems, compositions and methods for the processing of cereal grains, and in particular wheat, as well as optimized enzyme compositions suitable for that particular purpose.

### CROSS-REFERENCE TO RELATED APPLICATION

THIS application CLAIMS PRIORITY TO AND THE BENEFIT OF U.S. PROVISIONAL PATENT APPLICATION No. 62/128,066, TITLED “CEREAL GRAIN PROCESSING”, FILED Mar. 4, 2015.

### INCORPORATION BY REFERENCE OF THE SEQUENCE LISTING

The sequence listing provided in the file named “20160226_NB40804_PCT_ST25.txt” with a size of 20,539 bytes which was created on Feb. 26, 2016 and which is filed herewith, is incorporated by reference herein in its entirety.

### BACKGROUND

Milling is a process wherein wheat or the like is ground and pulverized to collect endosperm portions in a powder condition so that bran portions are not mixed with the endosperm portions. In the conventional milling process, after the initial cleaning steps, the wheat kernels are conditioned with water and/or steam and allowed to rest for more than 20 hours (tempering) to toughen the bran coats of the wheat kernels and soften the endosperm. Tempering of the wheat kernels fuses the bran coats together and is an essential conditioning step of the kernels carried out prior to the conventional milling process to alter the physical state of the kernels in a desired manner. In the conventional process, the tempering of the wheat kernels to toughen and to fuse the bran coats, unfortunately, also causes some fusion of the endosperm to the inner layers of bran whereby separation of these components is more difficult. Also traditional tempering or conditioning process steps require a long tempering time. The conditioned kernels are then subjected to successive stages, each of which grind, separate and purify the product. However, each grinding process produces fine bran particles (bran powder) and germ particles which have a tendency to be separated with the endosperm and are difficult, if not impossible, to remove from the endosperm. Each grinding operation produces more and more bran powder, compounding the problem. Effective removal of the bran from the endosperm remains a problem, which affects the yield possible from given wheat kernels as well as the color.

Continuous improvements of the milling processes and equipment have led to a steady improvement of extraction rates over the last centuries. Both the milling process and equipment have been improved during the past many years and consequently extraction rates have gone up. However, these measures have now reached their limits. In particular to change running systems without big investments is difficult. The methods of the present invention have solved many of the problems with the conventional milling and conditioning steps of the process.

### SUMMARY OF THE INVENTION

The present invention relates to compositions systems and methods for conditioning of cereal grains, e.g., compositions, methods and systems that have improved efficiency and are more cost effective. In one aspect, the present inventors disclose novel methods as well as novel enzyme compositions to solve the problems associated with conditioning of cereal (e.g. wheat). This not only provides more effective, time and cost effective conditioning, but also in terms of quality that may be seen in a final food product, such as bread.

In a first broad aspect the present invention relates to methods for the conditioning of a cereal grain, the method comprising the steps of conditioning cereal grains, such as wheat in the presence of a liquid composition comprising one or more cell-wall modifying enzyme.

In a second broad aspect the present invention relates to methods for the conditioning of a wheat grain, the method comprising the steps of: a) adding water in combination with a liquid composition comprising one or more cell-wall modifying enzyme(s) to the wheat grain; and b) conditioning the wheat grain for a specific amount of time for the wheat grain to absorb the water in the presence of said one or more cell-wall modifying enzyme(s). In some embodiments, the water is added to the wheat grain by spraying the water to the cereal grain. In some embodiments, the liquid composition is combined with the conditioning water using a dosing system.

Thus in some embodiments, the invention provides for a method for conditioning of wheat grain, the method comprising the steps of: a) spraying water in combination with a liquid composition comprising one or more cell-wall modifying enzyme(s) to the wheat grain; and b) Conditioning the wheat grain for a specific amount of time for the wheat grain to absorb the water in the presence of said one or more cell-wall modifying enzyme(s), wherein the liquid composition comprising the enzyme is combined with the conditioning water using a dosing system.

In a third aspect the present invention relates to methods for the extraction of flour from a cereal grain, the method comprising the steps of:

a) conditioning the cereal grain in a method according to the invention; and

b) milling the cereal grain and separate the flour from the bran of the cereal grain.

In a further aspect the present invention relates to a system suitable for operating a method according to the invention, wherein water containing one or more cell-wall modifying enzyme(s) is added to a composition of cereal grains during the conditioning of said cereal grains, the system containing a dosing system to adjust the amount of said enzyme being added to the cereal grains and a pump to mix said water containing one or more cell-wall modifying enzyme(s) with said composition of cereal grains.

In a further aspect the present invention relates to an aqua composition comprising an expression product obtained by fermentation of a species of the genus Trichoderma; which expression product comprises a beta-glucanase (EC 3.2.1.6) and a cellulase (EC 3.2.1.4), wherein said beta-glucanase is present in an amount of 1000-2000 AZO BBG U per gram aqua composition and said cellulase is present in an amount of 6000-8000 IU per gram aqua composition.

In a further aspect the present invention relates to an aqua composition comprising a xylanase (EC 3.2.1.8), wherein said xylanase is present in an amount of 100000-300000 units per gram aqua composition.

In a further aspect the present invention relates to the use of a system according to the invention, or an aqua composition according to the invention in a process of cereal grain conditioning.

In a further aspect the present invention relates to flour or cereal bran obtained from a method according to the invention or any food product obtained therefrom, such as a bread product.

Aspects and embodiments of the compositions and methods are set forth in the following separately numbered paragraphs.
1. A method for conditioning a cereal grain, the method comprising the steps of:
   a. providing a cereal grain comprising one or more β-glucans and one or more arabinoxylans;
   b. adding water in combination with a liquid composition comprising one or more cell-wall modifying enzyme(s) to the cereal grain; and
   c. conditioning the cereal grain for a specific amount of time for the cereal grain to absorb the water in the presence of said one or more cell-wall modifying enzyme(s).
      2. The method according to paragraph 1, wherein said cereal grain is wheat.
      3. The method according to any one of paragraphs 1 or 2, wherein said one or more cell-wall modifying enzyme(s) is selected from the group consisting of a xylanase, and a cellulase, such as cellobiohydrolases, beta-glucosidases, endo-glucanases, and beta-glucanase.
      4. The method according to paragraph 3, wherein the liquid composition further comprises one or more enzymes selected from the group consisting of arabinofuranosidase, xylosidase, mannanase, alpha-galactosidase, beta-glucuronidase, and beta-galactosidase.
      5. The method according to paragraph 3, wherein the liquid composition further comprises one or more enzymes selected from the group consisting of xylosidase, expansin-like and trypsin-like proteases.
      6. The method according to any one of paragraphs 1-5, wherein said conditioning is performed for more than 6 hours, such as for more than 8, 10, 12, 14, 16, 18, 20, 22, 24, 26, 28, 30, 32, 34, 36, 38, or 40 hours.
      7. The method according to any one of paragraphs 1-6, wherein said conditioning is performed for less than 40 hours, such as for less than 38, 36, 34, 32, 30, 28, 26, 24, 22, 20, 18, 16, 14, 12, 10, or 8 hours.
      8. The method according to any one of paragraphs 1-7, wherein said composition comprising one or more cell-wall modifying enzyme(s) is a liquid, such as an aqua formulation.
      9. The method according to any one of paragraphs 1-8, wherein said composition comprising one or more cell-wall modifying enzyme(s) is an aqua formulation comprising the enzymes secreted from the fermentation of the genus Trichoderma, such as Trichoderma reesei, such as beta-glucanases or cellulases.
      10. The method of paragraph 9 wherein said composition comprises one or more beta-glucanases and/or cellulases.
      11. The method of paragraph 10, wherein said composition further comprises one or more enzymes exhibiting xylanase activity.
      12. The method according to any one of paragraphs 10-11, wherein said composition further comprises one or more enzymes exhibiting beta-xylosidase activity.
      13. The method according to any one of paragraphs 10-12, wherein said composition further comprises one or more enzymes exhibiting mannanase activity.
      14. The method according to any one of paragraphs 10-13, wherein said composition further comprises one or more enzymes exhibiting arabinofuranosidase activity.
      15. The method according to any one of paragraphs 10-14, wherein said composition further comprises one or more enzymes exhibiting alpha-galactosidase activity
      16. The method according to any one of paragraphs paragraph 10-15, wherein said composition further comprises one or more enzymes exhibiting beta-glucuronidase activity.
      17. The method according to any one of paragraphs 10-16, wherein said composition further comprises one or more enzymes exhibiting beta-galactosidase activity.
      18. The method of paragraph 9, wherein said composition comprises one or more enzymes having an amino acid sequence having at least 80% identity to the enzymes selected from the group of enzymes having GenBank accession no. consisting of M16190, M15665, M19373, AB003694, Y11113, Z33381, AY281371, AY281372, AY281373, U09580, AB003110, AY281374, AY281375, AY281377, AY281378, AY281379, X69574, X69573, AB036796, Z69257, Z69256, AY281376, Z69252, AY281369, L25310, Z69253, Z69254, Z69255, Z68706, AJ549427, AJ245918, AY281370, AY281368, or any functional fragment thereof.
      19. The method of paragraph 9, wherein said composition comprises one or more enzymes having at least 80% identity to the enzymes selected from the group of enzymes having locus no. (from genome.jgi-psforg/Trire2/Trire2.home.html) of ORF_123283, ORF_76210, ORF_55319, ORF_54219, ORF_123989, ORF_123989, ORF_123989, ORF_123989, ORF_72567, ORF_72567, ORF_72567, ORF_72567, ORF_72567, ORF_72567, ORF_72567, ORF_72567, ORF_72567, ORF_72567, ORF_122081, ORF_120312, ORF_120312, ORF_123232, ORF_123232, ORF_49081, ORF_49081, ORF_49081, ORF_49081, ORF_27554, ORF_121127, ORF_121127, ORF_74223, ORF_123818, ORF_111849, ORF_56996, ORF_76672, and ORF_73897.
      20. The method of paragraphs 18-19 wherein said composition comprises two or more independently selected enzymes exhibiting beta-glucanase activity and at least one enzyme exhibiting xylanase activity.
      21. The method of paragraphs 18-20 wherein the one or more enzyme has at least 81, 82, 83, 84, 85, 86, 87, 88, 89, 90, 91, 92, 93, 94, 95, 96, 97, 98 or 99% identity with any one amino acid sequence.
      22. The method according to paragraph 9-21, wherein said beta-glucanase and said cellulase is present in an amount of 10,000 to 1,000,000 AZO BBG U per ton cereal grain (75,000-340,000), and 100,000 to 10×10⁶ IU per ton cereal grain (310,000-1,516,000) respectively.
      23. The method according to any one of paragraphs 1-22, wherein said composition comprising one or more cell-wall modifying enzyme(s) is an aqua formulation comprising the enzymes secreted from the fermentation of the genus bacillus, such as bacillus subtilis, such as a bacterial xylanase.
      24. The method according to any one of paragraphs 1-22, wherein said composition comprises one or more cell-wall modifying enzyme is an aqua formulation comprising an enzyme having xylanase activity, which enzyme comprises an amino acid sequence having at least 80% identity with any one of the amino acid sequences selected from SEQ ID NO:1-SEQ ID NO:8, or any functional fragment thereof.
      25. In The method according paragraph 24, wherein said composition comprises one enzymes comprising an amino acid sequence having at least 80% identity with any one of the amino acid sequences elected from SEQ ID NO:1; SEQ ID NO:2, SEQ ID NO:7 and SEQ ID NO:8, or any functional fragment thereof
      26. The method according to any one of paragraphs 1-22, further comprising one or more beta-glucanase.
      27. The method according to paragraph 23-26, wherein said xylanase is present in an amount of 1×10⁶ to 100×10⁶ units per ton cereal grain (9×10⁶ to 52×10⁶).
      28. The method according to any one of paragraphs 1-27, wherein said composition in step a further comprises one or more oxidase.
      29. The method of any preceding paragraph wherein, said adding water comprises spraying the cereal and wherein said one or more cell-wall modifying enzyme are added one or more times, or constantly, during said spraying.
      30. The method of any preceding paragraph, wherein the cereal reaches a moisture content of between about 12 to about 17%, and wherein said moisture content is reached in 12 hours or less.
      31. The method of any preceding paragraphs, wherein:
   (i) said cereal has 0.5-10% W/W of β-glucans; or
   (ii) said cereal has 1-10% W/W of arabinoxylans; or
   (iii) the amount of high molecular weight β-glucans in the cereal is decreased at least 50%; or
   (iv) the amount of high molecular weight β-glucans in the cereal is decreased at least 80%; or
   (v) the amount of high molecular weight arabinoxylans in the cereal is decreased at least 50%; or
   (vi) wherein the cereal is a hard cereal grain, and said liquid composition comprising one or more cell-wall modifying enzymes is added during the conditioning process at a concentration of 50-200 ppm for about 6 to 12 hours; or
   (vii) wherein the cereal is a mid-hard cereal grain, and said liquid composition comprising one or more cell-wall modifying enzymes is added during the conditioning process at a concentration of 50-200 ppm for about 6 to 12; or
   (viii) wherein the cereal is a soft cereal grain, and said liquid composition comprising one or more cell-wall modifying enzymes is added during the conditioning process at a concentration of 50-150 ppm for about 6 to 12; or
   (ix) wherein the cereal is a soft cereal grain, and said liquid composition comprising one or more cell-wall modifying enzymes is added during the conditioning process at a concentration of 50-150 ppm for about 6 or less.
      32. The methods of any preceding paragraph, wherein the amount of high molecular weight β-glucans in said cereal is no less than 150 mg/l, and the amount of arabinoxylans in said conditioned cereal is no less than 2000 mg/l. The methods of any preceding paragraph, wherein the amount of high molecular weight β-glucans in said cereal is no less than 50 mg/l, and the amount of arabinoxylans in said conditioned cereal is no less than 1000 mg/l.
      33. The method according to any preceding paragraph, said method comprises spraying water to said cereal, where said one or more cell wall modifying enzymes are added one or more times during said spraying, and wherein the amount of high molecular weight β-glucans in said conditioned cereal is no less 150 mg/l or less, and the amount of arabinoxylans in said cereal is at 2000 mg/l or less. The method according to any preceding paragraph, said method comprises spraying water to said cereal, where said one or more cell wall modifying enzymes are added one or more times during said spraying, and wherein the amount of high molecular weight β-glucans in said conditioned cereal is no less 50 mg/l or less, and the amount of arabinoxylans in said cereal is no less 1000 mg/l.
      34. The methods of any preceding paragraph, wherein the wet gluten in said conditioned cereal is at least 24%, 25%, 27%, 29%, 30%, 31% or 32%.
      35. A method for the extraction of flour from a cereal grain, the method comprising the steps of:
   a) conditioning the cereal grain in a method according to any one of paragraphs 1-17; and
   b) milling the cereal grain and separate the flour from the bran of the cereal grain.
      36. The method of paragraph 35, wherein the extraction rate increases at least about 0.5%, such as from at least about 0.6, 0.7, 0.8, 0.9, 1.0, 1.1, 1.2, 1.3, 1.4, 1.5, 1.6, 1.7, 1.8, 1.9, or 2% compared to a negative control cereal conditioned without said enzyme composition.
      37. System suitable for operating a method according to any one of paragraphs 1-19, wherein water containing one or more cell-wall modifying enzyme(s) is sprayed to a composition of cereal grains during the conditioning of said cereal grains, said system containing a dosing system to adjust the amount of said enzyme being added to said water containing one or more cell-wall modifying enzyme(s).
      38. The system according to paragraph 37 further comprising a mixing mechanism.
      39. An aqua composition comprising an expression product obtained by fermentation of a species of the genus Trichoderma; which expression product comprises a beta-glucanase (EC 3.2.1.6) and a cellulase (EC 3.2.1.4), wherein said beta-glucanase is present in an amount of 1000-2000 AZO BBG U per gram aqua composition and said cellulase is present in an amount of 6000-8000 IU per gram aqua composition.
      40. The aqua composition according to paragraph 39, wherein said expression product obtained by fermentation of the genus Trichoderma is from the species Trichoderma reesei.
      41. The aqua composition of paragraph 39, wherein said composition comprises one or more enzymes having an amino acid sequence having at least 80% identity to the enzymes selected from the group of enzymes having GenBank accession no. consisting of M16190, M15665, M19373, AB003694, Y11113, Z33381, AY281371, AY281372, AY281373, U09580, AB003110, AY281374, AY281375, AY281377, AY281378, AY281379, X69574, X69573, AB036796, Z69257, Z69256, AY281376, Z69252, AY281369, L25310, Z69253, Z69254, Z69255, Z68706, AJ549427, AJ245918, AY281370, AY281368, or any functional fragment thereof.
      42. The aqua composition of paragraph 39, wherein said composition comprises one or more enzymes having at least 80% identity to the enzymes selected from the group of enzymes having locus no. (from genome.jgi-psforg/Trire2/Trire2.home.html) of ORF_123283, ORF_76210, ORF_55319, ORF_54219, ORF_123989, ORF_123989, ORF_123989, ORF_123989, ORF_72567, ORF_72567, ORF_72567, ORF_72567, ORF_72567, ORF_72567, ORF_72567, ORF_72567, ORF_72567, ORF_72567, ORF_122081, ORF_120312, ORF_120312, ORF_123232, ORF_123232, ORF_49081, ORF_49081, ORF_49081, ORF_49081, ORF_27554, ORF_121127, ORF_121127, ORF_74223, ORF_123818, ORF_111849, ORF_56996, ORF_76672, and ORF_73897.
      43. The method of paragraphs 41-42 wherein said composition comprises two or more independently selected enzymes exhibiting beta-glucanase activity and at least one enzyme exhibiting xylanase activity.
      44. An aqua composition comprising a xylanase (EC 3.2.1.8), wherein said xylanase is present in an amount of 100000-300000 units per gram aqua composition.
      45. The aqua composition according to paragraph 44, comprising an expression product obtained by fermentation of a species of the genus Bacillus, such as a species Bacillus subtilis.
      46. The aqua composition according to paragraph 44, wherein said xylanase comprises an amino acid sequence having at least 80% identity with any one of the amino acid sequences selected from SEQ ID NO:1-SEQ ID NO:8, or any functional fragment thereof.
      47. The aqua composition according to paragraph 46, wherein said composition comprises one enzymes comprising an amino acid sequence having at least 80% identity with any one of the amino acid sequences elected from SEQ ID NO:1; SEQ ID NO:2, SEQ ID NO:7 and SEQ ID NO:8, or any functional fragment thereof
      48. The aqua composition according to any one of paragraphs 44-47, further comprising one or more beta-glucanase.
      49. Use of a system according to paragraph 37, or an aqua composition according to any one of paragraphs 39-48 in a process of cereal grain conditioning.
      50. Flour or cereal bran obtained from a method according to paragraph 35 or any food product obtained therefrom, such as a bread product.

### BRIEF DESCRIPTION OF THE BIOLOGICAL SEQUENCES

The following sequences comply with 37 C.F.R. §§ 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the European Patent Convention (EPC) and the Patent Cooperation Treaty (PCT) Rules 5.2 and 49.5 (a-bis), and Section 208 and Annex C of the Administrative Instructions. The symbols and format used for nucleotide and amino acid sequence data comply with the rules set forth in 37 C.F.R. § 1.822.

SEQ ID NO: 1 is the amino acid sequence of polypeptide AtuXyn3 having xylanase activity.
SEQ ID NO: 2 is the amino acid sequence of polypeptide TerXyn1 having xylanase activity.
SEQ ID NO: 3 is the amino acid sequence of polypeptide AtuXyn4 having xylanase activity.
SEQ ID NO: 4 is the amino acid sequence of polypeptide AacXyn2 having xylanase activity.
SEQ ID NO: 5 is the amino acid sequence of polypeptide TreXyn3 having xylanase activity.
SEQ ID NO: 6 is the amino acid sequence of polypeptide TreXyn5 having xylanase activity.
SEQ ID NO: 7 is the amino acid sequence of polypeptide BsuXyn3 having xylanase activity.
SEQ ID NO: 8 is the amino acid sequence of polypeptide BsuXyn4 having xylanase activity.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a flow chart of the milling process showing where enzymes are used in the milling process.

FIG. 2 shows the extraction rate for the German wheat KERUBINO. Trials with the wheat variety KERUBINO resulted in a significant higher extraction rate by reduced conditioning time.

FIG. 3 shows the energy savings when using enzymes. 12 h conditioning time was used for the reference while 6 h was used for the enzymes trials.

FIG. 4 shows baking trial with Argentinean low protein wheat flour.

FIG. 5 shows the baked breads of this baking trial.

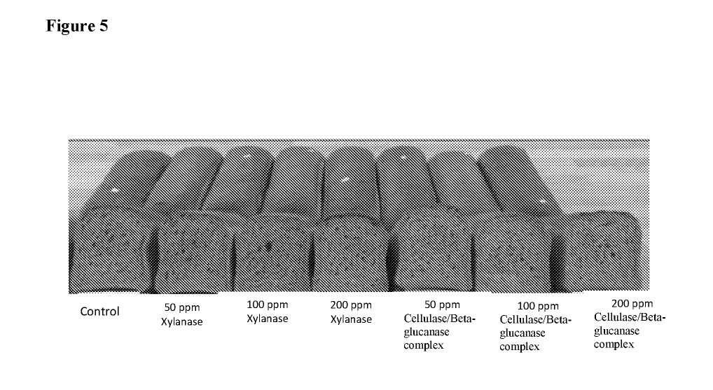

FIG. 6 shows baking trials of sandwich bread baked with whole meal flour.

### DETAILED DESCRIPTION OF THE INVENTION

The present disclosure provides composition, systems and methods for cereal processing, e.g., wheat conditioning. In some embodiments, the present invention relates to compositions systems and methods of processing of cereal grains, in particular wheat, for milling as well as optimized enzyme compositions suitable for that particular purpose. In some embodiments, the present invention provides systems, methods and compositions for improved processing of cereals, e.g., reduced conditioning time, reduce energy consumption and/or increase extraction rate. In some embodiments, the present invention provides systems, methods and compositions for improved conditioning of cereals which improve the milling processes.

In one aspect, the present invention is directed to composition and methods for the processing of cereals comprising adding one or more enzymes to the conditioning before milling. In some embodiments, the present invention provides compositions and methods for the processing of cereals comprising adding a cell wall degrading enzyme alone or in combination with other enzymes at some point during the conditioning before milling. In some embodiments, the cell wall degrading enzyme is a xylanase or a beta glucanase. In one embodiment, the present invention provides compositions and methods for the processing of cereals comprising adding one or more beta glucanases alone and/or one or more cellulases alone or in combination with other enzymes at some point during the conditioning before milling. In one embodiment, the present invention provides compositions and methods for the processing of cereals comprising adding a xylanase alone or in combination with other enzymes at some point during the conditioning before milling. In some embodiments, the other enzymes are other cell wall degrading enzymes. In some embodiments, the other enzymes are selected from the group consisting of arabinofuranosidase, xylosidase, mannanase, alpha-galactosidase, beta-glucuronidase, and beta-galactosidase. In some embodiments the invention provides compositions and methods for the processing of cereals comprising adding a beta glucanase/cellulase complex and/or a xylanase alone or in combination with other enzymes during the conditioning process. In some embodiments, the other enzymes are selected from the group consisting of arabinofuranosidase, xylosidase, mannanase, alpha-galactosidase, beta-glucuronidase, and beta-galactosidase.

Cereals, such as wheat, contain varying levels of β-glucans and arabinoxylans. (1,3;1,4)-β-D-Glucans consist of unbranched and unsubstituted chains of (1,3)- and (1,4)-β-glucosyl residues. The (1,3;1,4)-β-D-glucans are most abundant in cell walls of the cereals, specifically in the starchy endosperm of grain, where they can contribute up to 70% by weight of the cell walls. Arabinoxylan is a hemicellulose found in both the primary and secondary cell walls of cereal grains, consisting of copolymers of two pentose sugars—arabinose and xylose. The arabinose moiety may be further substituted.

Studies have shown that the content of β-glucans and arabinoxylans in the cell wall might be related with grain hardness as well as water uptake of the cereals (e.g. wheat). Kernel harness is one of the major factors affecting the processing and product quality of the grain. A high β-glucans and arabinoxylans content in the cereal may lead to insufficient degradation of cell walls, which in turns may affect the milling process, and hence reduces the extraction rate.

Without intending to be limited to any theory, in some embodiments, the present invention provides compositions and methods to breakdown betaglucans and other cell wall components such as arabinoxylans in the grains during the conditioning process. By breaking down the betaglucans and other cell wall components (e.g., arabinoxylans) in the grain during the conditioning process the present invention allows for reduction in conditioning time, improvement in extraction rate, energy savings, alignment and shortening of conditioning time (even when blending different grains with different humidification needs), no change to the flour quality or the baking performance (i.e., same high quality), reduced loss during milling, and/or increased whitening of the flour and more integral bran. In addition, by decreasing the water binding capacity of beta-glucans and arabinoxylans in the cell wall of the grain, the methods, systems and compositions described herein allow a better and faster water uptake by the grain.

In a typical milling process, the mill receives the grains such as wheat and sends it through cleaning. The next step is then the conditioning process, where the grains, such as wheat is humidified, e.g., by spraying water. In some embodiments, one or more enzymes described herein are adding during the conditioning process, e.g., by combining the enzyme compositions with the conditioning water. In some embodiments, the conditioning process comprises spraying water containing the enzymes compositions described herein to the cereal grains. The conditioning process may take from 4-40 hours depending on wheat and process, before the wheat is then milled and extracted to flour and bran.

“Conditioning”, “tempering” or “damping” are all terms used to describe this part of the process of adding water to cereal grains to allow extraction of flour and to ensure that quality parameters can be met.

The conditioning softens the outer pericarp (bran) layer of the grain, such as wheat and enhances the release of the inner white endosperm during milling. It also helps to soften the grain by softening the starchy structures of the endosperm. The amount of water added at this stage is dependent on several factors: (i) The variety of grain; (ii) Grain hardness; (iii) The natural moisture content; (iv) the milling process; and (v) Specification of the finished flour.

In some embodiments, the present invention is directed to systems composition and methods for the processing of wheat comprising adding one or more enzymes to the conditioning before milling.

Normal moisture levels in wheat vary from around 9% up to 14% dependent on variety and climatic conditions during harvest. Humidity normally needs to be raised to 15-17% prior to milling. Conditioning time varies between 4 h-40 h to allow moisture to penetrate evenly through the grain. Typically this time is dictated by grain variety and is shorter for soft wheat and longer for hard wheat.

Conditioning time has a direct impact on the efficiency of a flour mill and requires large storage space. The reduction of conditioning time made possible by the systems, compositions and methods described herein, is therefore significant in improving the efficiency of mill operations. Without intending to be limited to any theory, the compositions and methods described herein open the structure of the grain and reduce the water binding capacity of several cell wall components, as a result, water penetrates the grain easier and more quickly. The Examples show that it is possible to reduce conditioning time by 30-50% dependent and/or reduce the energy needed to process the grains. This provides benefits for the millers such as higher flexibility in the milling process, reduced storage space and faster time of response for customer demands.

In some embodiments, the systems, compositions and methods according to the present invention provide better flour as well as bran in terms of quality. The grain such as wheat is leaving during process almost unbroken so that flours is getting significantly less pigmentation from bran as has been seen with known processes. Also the addition of water influences the whitening of the flour.

The term, “cereal grain” as used herein refers to the fruits from a plant of the family Poaceae, such seed containing at least the bran comprising the aleurone, and the starchy endosperm, with the additional presence of pericarp, seed coat (alternatively called testa) and/or germ. The term includes, but is not limited to species such as wheat, barley, oat, spelt, rye, sorghum, maize, and rice.

The term, “conditioning” as used herein refers to the stage wherein the cereal grain such as wheat is allowed to absorb water.

The terms “bran” as used herein refers to a cereal-derived milling fraction enriched in any or all of the tissues to be selected from aleurone, pericarp and seed coat, as compared to the corresponding intact seed.

The term “milling fraction”, as used herein, refers to all or part of the fractions resulting from mechanical reduction of the size of grains, through, as examples but not limited to, cutting, rolling, crushing, breakage or milling, with or without fractionation, through, as examples but not limited to, sieving, screening, sifting, blowing, aspirating, centrifugal sifting, windsifting, electrostatic separation, or electric field separation

### Enzymes

In one aspect, the present invention is directed to systems, compositions and methods for the processing of cereals comprising adding one or more enzymes to the conditioning process prior to milling. In one embodiment, the present invention provides compositions and methods for the processing of cereals comprising adding one or more beta glucanases or cellulases and/or one or more xylanases alone or in combination with other enzymes during the conditioning process prior to milling.

In one embodiment, the present invention provides compositions and methods for the processing of cereals comprising adding one or more beta glucanases and/or one or more cellulases alone or in combination with other enzymes during the conditioning process prior to milling. In one embodiment, the present invention provides compositions and methods for the processing of cereals comprising adding one or more xylanases alone or in combination with other enzymes during the conditioning process prior to milling. In some embodiments, the other enzymes are selected from the group consisting of arabinofuranosidase, xylosidase, mannanase, alpha-galactosidase, beta-glucuronidase, and beta-galactosidase.

In some embodiments, enzyme compositions according to the present invention are produced by using selected strains of bacteria and/or fungi.

In the context of the present invention, “cell-wall modifying enzyme”, refers to any enzyme capable of hydrolyzing or modifying the complex matrix polysaccharides of the plant cell wall, such as any enzyme that will have activity in the “cell wall solubilisation assay” included herein. Included within this definition of “cell-wall modifying enzyme” are cellulases, such as cellobiohydrolase I and cellobiohydrolase II, endo-glucanases and beta-glucosidases, and hemicellulolytic enzymes, such as xylanases.

The terms “cellulases” or “cellulolytic enzymes” as used herein are understood as comprising the cellobiohydrolases (EC 3.2.1.91), e.g., cellobiohydrolase I and cellobiohydrolase II, as well as the endo-glucanases (EC 3.2.1.4) and beta-glucosidases (EC 3.2.1.21).

Included with the definition of cellulases are: endoglucanases (EC 3.2.1.4) that cut the cellulose chains at random; cellobiohydrolases (EC 3.2.1.91) which cleave cellobiosyl units from the cellulose chain ends and beta-glucosidases (EC 3.2.1.21) that convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is defined herein as a cellulose 1,4-beta-cellobiosidase (also referred to as exo-glucanase, exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase I1 activity” is identical, except that cellobiohydrolase I1 attacks from the reducing ends of the chains.

The cellulases may comprise a carbohydrate-binding module (CBM) which enhances the binding of the enzyme to a cellulose-containing fiber and increases the efficacy of the catalytic active part of the enzyme. A CBM is defined as contiguous amino acid sequence within a carbohydrate-active enzyme with a discreet fold having carbohydrate-binding activity. For further information of CBMs see the CAZy internet server (Supra) or Tomme et al. (1995) in Enzymatic Degradation of Insoluble Polysaccharides (Saddler and Penner, eds.), Cellulose-binding domains: classification and properties, pp. 142-163, American Chemical Society, Washington. In a preferred embodiment the cellulases or cellulolytic enzymes may be a cellulolytic preparation as defined in U.S. application No. 60/941,251, which is hereby incorporated by reference. In a preferred embodiment the cellulolytic preparation comprising a polypeptide having cellulolytic enhancing activity (GH61A), preferably the one disclosed in WO 2005/074656. The cell-wall modifying enzyme may further be a beta-glucosidase, such as a beta-glucosidase derived from a strain of the genus Trichoderma, Aspergillus or Penicillium, including the fusion protein having beta-glucosidase activity disclosed in U.S. application No. 60/832,511 (Novozymes). In some embodiments the cell-wall modifying enzyme is a CBH II, such as Thielavia terrestris cellobiohydrolase I1 (CEL6A). In some embodiments the cell-wall modifying enzyme is a cellulase enzyme, such as one derived from Trichoderma reesei.

The cellulolytic activity may, in some embodiments, be derived from a fungal source, such as a strain of the genus Trichoderma, such as a strain of Trichoderma reesei; or a strain of the genus Humicola, such as a strain of Humicola insolens.

In some embodiments the cell-wall modifying enzyme is a polypeptide having cellulolytic enhancing activity (GH61A) disclosed in WO 2005/074656; a cellobiohydrolase, such as Thielavia terrestris cellobiohydrolase I1 (CEL6A), a beta-glucosidase (e.g., the fusion protein disclosed in U.S. application No. 60/832,511) and cellulolytic enzymes, e.g., derived from Trichoderma reesei.

In some embodiments the cell-wall modifying enzyme is a polypeptide having cellulolytic enhancing activity (GH61A) disclosed in WO 2005/074656; a beta-glucosidase (e.g., the fusion protein disclosed in U.S. application No. 60/832,511) and cellulolytic enzymes, e.g., derived from Trichoderma reesei. In some embodiments the cell-wall modifying enzyme is a commercially available product, such as GC220 available from Genencor, A Danisco Division, US or CELLUCLAST® 1.5L or CELLUZYME™ available from Novozymes A/S, Denmark.

Endoglucanases (EC No. 3.2.1.4) catalyses endo hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification. Endoglucanase activity may be determined using carboxymethyl cellulose (CMC) hydrolysis according to the procedure of Ghose, 1987, Pure and Appl. Chem. 59: 257-268.

In some embodiments endoglucanases may be derived from a strain of the genus Trichoderma, such as a strain of Trichoderma reesei; a strain of the genus Humicola, such as a strain of Humicola insolens; or a strain of Chrysosporium, preferably a strain of Chrysosporium lucknowense.

The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain.

Examples of cellobiohydroloses are mentioned above including CBH I and CBH I1 from Trichoderma reesei; Humicola insolens and CBH I1 from Thielavia tenrestris cellobiohydrolase (CELL6A).

Cellobiohydrolase activity may be determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288. The Lever et al. method is suitable for assessing hydrolysis of cellulose in corn stover and the method of van Tilbeurgh et al., is suitable for determining the cellobiohydrolase activity on a fluorescent disaccharide derivative.

The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, J. Basic Microbiol. 42: 55-66, except different conditions were employed as described herein. One unit of beta-glucosidase activity is defined as 1.0 ?mole of p-nitrophenol produced per minute at 500 C, pH 5 from 4 mM p-nitrophenyl-beta-D-glucopyranoside as substrate in 100 mM sodium citrate, 0.01% TWEEN® 20.

In some embodiments the beta-glucosidase is of fungal origin, such as a strain of the genus Trichoderma, Aspergillus or Penicillium. In some embodiments the beta-glucosidase is a derived from Trichoderma reesei, such as the beta-glucosidase encoded by the bgl1 gene (see EP 562003). In another embodiment the beta-glucosidase is derived from Aspergillus oryzae (recombinantly produced in Aspergillus oryzae according to WO 02/095014), Aspergillus fumigatus (recombinantly produced in Aspergillus oryzae according to Example 22 of WO 02/095014) or Aspergillus niger (1981, J. Appl. 3: 157-163).

The terms “hemicellulolvtic enzymes” or “hemicellulases”, as used herein, refers to enzymes that may break down hemicellulose.

Any hemicellulase suitable for use in hydrolyzing hemicellulose, preferably into arabinoxylan oligosaccharides, may be used. Preferred hemicellulases include xylanases, arabinofuranosidases, acetyl xylan esterase, feruloyl esterase, glucuronidases, galactanase, endo-galactanase, mannases, endo or exo arabinases, exo-galactanses, pectinase, xyloglucanase, or mixtures of two or more thereof. An example of hemicellulase suitable for use in the present invention includes Grindamyl Powerbake 930 (available from Danisco A/S, Denmark) or VISCOZYM E™ (available from Novozymes A/S, Denmark). In an embodiment the hemicellulase is a xylanase. In an embodiment the xylanase is of microbial origin, such as of fungal origin (e.g., Trichoderma, Meripilus, Humicola, Aspergillus, and Fusarium) or from a bacterium (e.g., Bacillus). In some embodiments the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH 10 or GH11. Examples of commercial xylanases include Grindamyl H121 or Grindamyl Powerbake 930 from Danisco A/S, Denmark or SHEARZYIVIE™ and BIOFEED WHEAT™ from Novozymes A/S, Denmark.

Arabinofuranosidase (EC 3.2.1.55) catalyzes the hydrolysis of terminal non-reducing alpha-L-arabinofuranoside residues in alpha-L-arabinosides. Galactanase (EC 3.2.1.89), arabinogalactan endo-1,4-beta-galactosidase, catalyses the endohydrolysis of 1,4-D-galactosidic linkages in arabinogalactans.

Pectinase (EC 3.2.1.15) catalyzes the hydrolysis of 1,4-alpha-D-galactosiduronic linkages in pectate and other galacturonans.

Xyloglucanase catalyzes the hydrolysis of xyloglucan.

The term “xylanase” as used herein refers to an enzyme that is able to hydrolyze the beta-1,4 glycosyl bond in non-terminal beta-D-xylopyranosyl-1,4-beta-D-xylopyranosyl units of xylan or arabinoxylan. Other names include 1,4-beta-D-xylan xylanohydrolase, 1,4-beta-xylan xylanohydrolase, beta-1,4-xylan xylanohydrolase, (1-4)-beta-xylan 4-xylanohydrolase, endo-1,4-beta-xylanase, endo-(1-4)-beta-xylanase, endo-beta-1,4-xylanase, endo-1,4-beta-D-xylanase, endo-1,4-xylanase, xylanase, beta-1,4-xylanase, beta-xylanase, beta-D-xylanase. Xylanases can be derived from a variety of organisms, including plant, fungal (e.g. species of Aspergillus, Penicillium, Disporotrichum, Neurospora, Fusarium, Humicola, Trichoderma) or bacterial species (e.g. species of Bacillus, Aeromonas, Streptomyces, Nocardiopsis, Thermomyces) (see for example WO92/17573, WO92/01793, WO91/19782, WO94/21785). In some aspects of the invention, the xylanase is as specifically disclosed in any one of WO 2010/072224, WO 2010/072225, WO 2010/072226 and WO0166711.

In one aspect of the invention, the xylanase used in the methods of the invention is an enzyme classified as EC 3.2.1.8. The official name is endo-1,4-beta-xylanase. The systematic name is 1,4-beta-D-xylan xylanohydrolase. Other names may be used, such as endo-(1-4)-beta-xylanase; (1-4)-beta-xylan 4-xylanohydrolase; endo-1,4-xylanase; xylanase; beta-1,4-xylanase; endo-1,4-xylanase; endo-beta-1,4-xylanase; endo-1,4-beta-D-xylanase; 1,4-beta-xylan xylanohydrolase; beta-xylanase; beta-1,4-xylan xylanohydrolase; endo-1,4-beta-xylanase; beta-D-xylanase. The reaction catalyzed is the endohydrolysis of 1,4-beta-D-xylosidic linkages in xylans.

In one aspect of the invention, the xylanase of the invention is a xylanase of Glycoside Hydrolyase (GH) Family 11. The term “of Glycoside Hydrolyase (GH) Family 11” means that the xylanase in question is or can be classified in the GH family 11.

In one aspect of the invention, the xylanase used according to the invention, is a xylanase having xylanase activity as measured in the “Xylanase assay” as described herein.

According to the Cazy(ModO) site, Family 11 glycoside hydrolases can be characterised as follows: (i) Known Activities: xylanase (EC 3.2.1.8); (ii) Mechanism: Retaining; (iii) Catalytic Nucleophile/Base: Glu (experimental); (iv) Catalytic Proton Donor: Glu (experimental); (v) 3D Structure Status: Fold:—jelly roll; and (vi) Clan: GH-C

As used herein, “Clan C” refers to groupings of families which share a common three-dimensional fold and identical catalytic machinery (see, for example, Henrissat, B. and Bairoch, A., (1996) Biochem. J., 316, 695-696).

As used herein, “Family 11” refers to a family of enzymes as established by Henrissat and Bairoch (1993) Biochem J., 293, 781-788 (see, also, Henrissat and Davies (1997) Current Opinion in Structural Biol. 1997, &:637-644). Common features for family 11 members include high genetic homology, a size of about 20 kDa and a double displacement catalytic mechanism (see Tenkanen et al., 1992; Wakarchuk et al., 1994). The structure of the family 11 xylanases includes two large beta-sheets made of beta-strands and beta-helices.

Family 11 xylanases include the following: Aspergillus niger XynA, Aspergillus kawachii XynC, Aspergillus tubingensis XynA, Bacillus circulans XynA, Bacillus punzilus XynA, Bacillus subtilis XynA, Neocalliniastix patriciarum XynA, Streptomyces lividans XynB, Streptomyces lividans XynC, Streptomyces therinoviolaceus XynII, Thermomonospora fusca XynA, Trichoderma harzianum Xyn, Trichoderma reesei XynI, Trichoderma reesei XynII, Trichoderma viride Xyn.

In further aspects, the enzyme for the methods and compositions described herein has laminarinase activity or comprises any one or more further enzyme having laminarinase activity. The laminarinase activity can be determined as described in the laminarinase assays herein or by any feasible method known in the art.

Laminarinase may be an endo-1,3(4)-beta-glucanase classified in E.C. 3.2.1.6 or glucan endo-1,3-beta-D-glucosidase classified in E.C. 3.2.1.39. Endo-1,3(4)-beta-glucanase with the alternative names, laminarinase, endo-1,3-beta-glucanase. Endo-1,4-beta-glucanase is classified in E.C. 3.2.1.6. The substrates include laminarin, lichenin and cereal D-glucans. The enzyme catalyzes endohydrolysis of (1→3)- or (1→4)-linkages in beta-D-glucans when the glucose residue whose reducing group is involved in the linkage to be hydrolyzed is itself substituted at C-3. Glucan endo-1,3-beta-D-glucosidase with the alternative names (1→3)-beta-glucan endohydrolase, Endo-1,3-beta-glucanase and laminarinase is classified in E.C. 3.2.1.39. Glucan endo-1,3-beta-D-glucosidase hydrolyses (1→3)-beta-D-glucosidic linkages in (1→3)-beta-D-glucans in substrates as e.g. laminarin, paramylon and pachyman.

In some aspects, the enzyme for the methods and compositions described herein has xyloglucan-specific exo-beta-1,4-glucanase activity or comprises a further enzyme having xyloglucan-specific exo-beta-1,4-glucanase activity, “xyloglucan-specific exo-beta-1,4-glucanase” refers to enzymes of E.C3.2.1.155. Xyloglucan-specific exo-beta-1,4-glucanase catalyze the exohydrolysis of (1→4)-beta-D-glucosidic linkages in xyloglucan.

In some aspects, the enzyme composition according to the invention has alpha-N-arabinofuranosidase activity or comprises a further enzyme having arabinofuranosidase activity. “alpha-N-arabinofuranosidase” or “Alpha-N-arabinofuranosidase” refers to enzymes of EC 3.2.1.55. alpha-N-arabinofuranosidase catalyzes the hydrolysis of terminal non-reducing alpha-L-arabinofuranoside residues in alpha-L-arabinosides.

In one aspect of the invention, the arabinofuranosidase activity of the enzyme composition according to the invention is measured by the arabinofuranosidase assay as described herein or by any suitable assay known in the art. The standard assay can be carried out at pH 5.0 and 50° C. and it can be performed at different values of pH and temperatures for the additional characterization and specification of enzymes.

One unit of alpha-N-arabinofuranosidase activity is defined as the amount of enzyme which produces 1 μmole p-nitrophenol from p-nitrophenyl alpha-L-arabinofuranoside per minute under the conditions of the assay (e.g., pH 5.0 and 50° C. (or as specified)).

In some aspects, the enzyme composition according to the invention has glucan 1,4-betaglucosidase activity or comprises a further enzyme having glucan 1,4-beta-glucosidase activity. “Glucan 1,4-beta-glucosidase” or “glucan 1,4-beta-glucosidase” refers to enzymes of E.C3.2.1.74. Glucan 1,4-beta-glucosidase catalyze the hydrolysis of (1→4)-linkages in (1→4)-beta-D-glucans, to remove successive glucose units.

In some embodiments the one or more cell-wall modifying enzyme used in the methods or compositions according to the present invention is a wild type enzyme or an enzyme complex containing a plurality of different enzyme activities produced upon fermentation of one or more selected strains of a bacteria or fungus, such as a strain of the genus Trichoderma, or a strain of the genus bacillus. This may include wild type activities of a xylanase (EC 3.2.1.8), a beta-glucanase (EC 3.2.1.6) and/or a cellulase (EC 3.2.1.4). In some embodiments the one or more cell-wall modifying enzyme used in the methods or compositions according to the present invention is an expression product obtained by fermentation of a species of the genus Trichoderma, such as Trichoderma reesei. In some embodiments the one or more cell-wall modifying enzyme used in the methods or compositions according to the present invention is an expression product obtained by fermentation of a species of the genus bacillus, such as bacillus subtilis.

In some embodiments the one or more cell-wall modifying enzyme used in the methods or compositions according to the present invention is an enzyme complex containing a plurality of different enzyme activities produced upon fermentation of Trichoderma reesei selected from the group consisting of cellobiohydrolase, endo-1,4-glucanase, beta-glucosidase, xylanase, beta-xylosidase, acetyl xylan esterase, arabinofuranosidase, beta-mannanase, alpha-galactosidase, alpha-glucuronidase, beta-galactosidase and expansin-like. In some embodiments, the plurality of enzymes are selected from the group of enzymes having GenBank accession no. consisting of M16190, M15665, M19373, AB003694, Y11113, Z33381, AY281371, AY281372, AY281373, U09580, AB003110, AY281374, AY281375, AY281377, AY281378, AY281379, X69574, X69573, AB036796, Z69257, Z69256, AY281376, Z69252, AY281369, L25310, Z69253, Z69254, Z69255, Z68706, AJ549427, AJ245918, AY281370, AY281368. In some embodiments, the enzyme complex comprises at least 2, or at least 3, or at least 4, or at least 5, or at least 7, or at least 8, or at least 10, or at least 15, or at least 20 of the enzymes listed above. In some embodiments, the enzyme complex comprises up to 5, or up to 8, or up to 10, or up to 15, or up to 20, or all the enzymes listed above. In some embodiments, the enzyme complex comprise a plurality of different enzyme activities having an amino acid sequence having at least 80% identity to the plurality of enzymes described above. In some embodiments, the one or more enzyme has at least 81, 82, 83, 84, 85, 86, 87, 88, 89, 90, 91, 92, 93, 94, 95, 96, 97, 98 or 99% identity with any one amino acid sequence described above.

In some embodiments the one or more cell-wall modifying enzyme used in the methods or compositions according to the present invention is an enzyme complex containing a plurality of different enzyme activities produced upon fermentation of Trichoderma reesei selected from the group consisting of Arabinofuranosidase, Candidate acetyl xylan esterase, Cellobiohydrolase I, Cellobiohydrolase II, Endoglucanase I, Endoglucanase II, Endoglucanase III, Xyloglucanase, Candidate Endoglucanase, Xylosidase I, Xylanase I, Xylanase II, Xylanase IV, Mannanase I, β-Glucosidase, and Trypsin-like protease. In some embodiments, the plurality of enzymes are selected from the group of enzymes having locus no. (from genome.jgi-psf.org/Trire2/Trire2.home.html) consisting of ORF_123283, ORF_76210, ORF_55319, ORF_54219, ORF_123989, ORF_123989, ORF_123989, ORF_123989, ORF_72567, ORF_72567, ORF_72567, ORF_72567, ORF_72567, ORF_72567, ORF_72567, ORF_72567, ORF_72567, ORF_72567, ORF_122081, ORF_120312, ORF_120312, ORF_123232, ORF_123232, ORF_49081, ORF_49081, ORF_49081, ORF_49081, ORF_27554, ORF_121127, ORF_121127, ORF_74223, ORF_123818, ORF_111849, ORF_56996, ORF_76672, and ORF_73897. In some embodiments, the enzyme complex comprises at least 2, or at least 3, or at least 4, or at least 5, or at least 7, or at least 8, or at least 10, or at least 15, or at least 20 of the enzymes listed above. In some embodiments, the enzyme complex comprises up to 5, or up to 8, or up to 10, or up to 15, or up to 20, or all the enzymes listed above. In some embodiments, the enzyme complex comprise a plurality of different enzyme activities having an amino acid sequence having at least 80% identity to the plurality of enzymes described above. In some embodiments, the one or more enzyme has at least 81, 82, 83, 84, 85, 86, 87, 88, 89, 90, 91, 92, 93, 94, 95, 96, 97, 98 or 99% identity with any one amino acid sequence described above.

In some embodiments, the enzyme has a total number of amino acids of less than 350, such as less than 340, such as less than 330, such as less than 320, such as less than 310, such as less than 300 amino acids, such as in the range of 200 to 350, such as in the range of 220 to 345 amino acids than the one or more enzyme described herein.

In some embodiments, the amino acid sequence of the enzyme has at least one, two, three, four, five, six, seven, eight, nine or ten amino acid substitutions.

In some embodiments, one or more enzyme exhibiting cellulase activity and/or one or more enzymes exhibiting beta-glucanase activity, and/or one or more enzymes exhibiting xylanase activity, alone or in combination, provide for improved conditioning processes before milling.

In some embodiments, one or more enzyme exhibiting cellulase activity and/or one or more enzymes exhibiting beta-glucanase activity, and/or one or more enzymes exhibiting xylanase activity, alone or in combination, provide for opening of the wheat kernel structure during conditioning of the grain with water, which enables water to migrate faster between the different layers. This leads to shorter conditioning time, easier separation of the endosperm from the aleurone layer and results in a potentially higher extraction rate

In some embodiments, one or more enzyme exhibiting cellulase activity and/or one or more enzymes exhibiting beta-glucanase activity, and/or one or more enzymes exhibiting xylanase activity, alone or in combination, provide for reduce conditioning time (e.g. by 30-50%).

In some embodiments, one or more enzyme exhibiting cellulase activity and/or one or more enzymes exhibiting beta-glucanase activity, and/or one or more enzymes exhibiting xylanase activity, alone or in combination, provide for increase extraction rates (e.g., from 0.5%-2%).

In some embodiments, one or more enzyme exhibiting cellulase activity and/or one or more enzymes exhibiting beta-glucanase activity, and/or one or more enzymes exhibiting xylanase activity, alone or in combination, provide for energy savings (e.g. up to 20%, e.g. from 5 to 20%).

In some embodiments, one or more enzyme exhibiting cellulase activity and/or one or more enzymes exhibiting beta-glucanase activity, and/or one or more enzymes exhibiting xylanase activity, alone or in combination, provide for whitening effect in the flour.

In some embodiments, one or more enzyme exhibiting cellulase activity and/or one or more enzymes exhibiting beta-glucanase activity, and/or one or more enzymes exhibiting xylanase activity, alone or in combination, provide for stable flour quality.

One aspect of the invention relates to an enzyme exhibiting xylanase activity, which enzyme comprises an amino acid sequence having at least 80% identity with any one selected from SEQ ID NO:1-SEQ ID NO:8, or any functional fragment thereof. In some embodiments, the invention relates to an enzyme exhibiting xylanase activity, which enzyme comprises an amino acid sequence having at least 80% identity with any one selected from SEQ ID NO:1; SEQ ID NO:2, SEQ ID NO:7 and SEQ ID NO:8, or any functional fragment thereof.

As used herein “functional fragment” refers to a truncated version of an enzyme with essentially the same or at least a significant degree of enzyme activity as the non-truncated reference enzyme.

In some embodiments the composition and methods according to the invention comprises the use of any suitable commercially available enzymes having xylanase activity and/or beta-glucanase activity such as UltraFlo L (available from Novozymes—beta glucanase with cellulase, xylanase side activities), UltraFlo XL (available from Novozymes—beta glucanase with xylanase and alpha amylase side activities), UltraFlo Max (available from Novozymes—beta glucanase and xylanase), Finizyme 250 L (available from Novozymes—beta glucanase with cellulase, xylanase side activities) Filtrase series (available from DSM-beta glucanase and xylanases).

In some embodiments, the enzyme has a temperature optimum in the range of 5-80° C., such as in the range of 5-40° C. or 15-80° C., such as in the range 20-80° C., such as in the range 5-15° C., 15-20° C., 45-65° C., 50-65° C., 55-65° C. or 60-80° C.

Sequences and enzymes identified by a sequence as mentioned herein and used according to the present invention alone or in combinations with other enzymes or compounds may be with or without signal peptide.

In one embodiment the enzyme(s) may be secreted from the fermentation of an organism in the genus Trichoderma, such as Trichoderma reesei. The enzymes may be a beta-glucanase or cellulose for example.

The Trichoderma (or Trichoderma reesei) may be fermented according to known fermentation methods. By way of example only, fermentation conditions may be according to those detailed in Ross et al European J. of Applied Microbiology and Biotechnology January 1983, Vol. 18, Issue 1, pp 27-37 (which is herein incorporated by reference).

In one embodiment the Trichoderma (or Trichoderma reesei) may be fermented in any suitable nutrient media. By way of example only, the composition of the nutrient medium may include the following constituents: (NH₄)₂SO₄ (e.g. at 3.0 g/l); KH₂PO₄ (e.g. at 2 g/l), CaCl₂) (e.g. at 0.3 g/l), MgSO₄ (e.g. at 0.3 g/l), Yeast extract (e.g. at 1 g/l), FeSO₄, 7H₂O (e.g. at 5 mg/l), MnSO₄ (e.g. at 1.56 mg/l), ZnSO₄, 7H₂O (e.g. at 1.4 mg/l), CoCl₂ (e.g. at 2 mg/l), cellulose (e.g. Avicel cellulose) (e.g. at 10 g/l).

The fermentation in Trichoderma (or Trichoderma reesei) may be performed using suitably conditions. For example, the fermentation in Trichoderma (or Trichoderma reesei) may be performed with growth at about 25° C. to about 37° C., advantageously at about 34° C., and about pH 3 to about pH 5.5, advantageously about pH 3.5, and with production at about 25° C. to 30° C., advantageously at about 28° C. and about pH 3 to about pH 6, advantageously at about pH 4.5. Antifoam may need to be added. The enzyme(s) are typically secreted into the fermentation broth. After fermentation the broth may be filtered (e.g. on a rotary vacuum drum filter) to remove the cells of the bacteria. The clear liquid may be further subjected to ultrafiltration to concentrate and purify the enzyme(s). Further microfiltration may be undertaken. The enzyme concentrate may be formulated, e.g. as a liquid preparation.

In one embodiment the enzyme(s) (e.g. a xylanase) may be secreted from the fermentation of bacteria from the genus Bacillus, such as Bacillus subtilis.

The Bacillus (or Bacillus subtilis) may be fermented according to known fermentation methods. By way of example only, fermentation conditions may be according to those detailed in Olempska-Beer, Chemical and Technical Assessment 63^{rd} JECFA 2004.

The enzymes secreted from the fermentation of Bacillus (or Bacillus subtilis) may be produced by submerged fermentation using a fermentation medium composed of food grade materials. The enzymes are typically secreted into the fermentation broth. After fermentation the broth may be filtered (e.g. on a rotary vacuum drum filter) to remove the cells of the bacteria. The clear liquid may be further subjected to ultrafiltration to concentrate and purify the enzyme(s), e.g. the xylanase. Further microfiltration may be undertaken. The enzyme concentrate may be formulated, e.g. as a liquid preparation.

In one embodiment the Bacillus (or Bacillus subtilis) may be fermented in any suitable nutrient media. By way of example only, the composition of the nutrient medium may include the following constituents: polypeptone (e.g. at 1%) or glucose (e.g. at 1%); KH₂PO₄ (e.g. at 0.1%), NaCl₂ (e.g. at 0.3%), MgSO₄ 7H₂O (e.g. at 0.02%), Yeast extract (e.g. at 0.5%), xylan (e.g. oat spelt xylan) (e.g. at 0.5%) and Na₂CO₃ (e.g. at 1%).

The fermentation in Bacillus (or Bacillus subtilis) may be performed at about 37° C. to 55° C., and about pH 6.5 to about pH 9.0. Suitably the fermentation may be agitated (e.g. at about 400 rpm). Suitably the fermentation may comprise a dissolved oxygen concentration of about 5-6.5 mg/l, suitably 6.0 mg/l.

### Compositions

In one aspect, the present disclosure provides compositions for cereal processing, e.g., wheat conditioning. In some embodiments, the present invention relates to compositions for processing of cereal grains, in particular wheat, for milling as well as optimized enzyme compositions suitable for that particular purpose. In some embodiments, the present invention provides compositions for improved processing of cereals, e.g., reduced conditioning time, reduce energy consumption and/or increase extraction rate. In some embodiments, the present invention compositions for improved conditioning of cereals which improve the milling processes.

In one aspect, the present invention is directed to composition for the processing of cereals comprising one or more enzymes to the conditioning before milling. In some embodiments, the present invention provides compositions for the processing of cereals comprising a cell wall degrading enzyme alone or in combination with other enzymes at some point during the conditioning before milling. In some embodiments, the cell wall degrading enzyme is a xylanase or a beta glucanase. In one embodiment, the present invention provides compositions for the processing of cereals comprising adding one or more beta glucanases alone and/or one or more cellulases alone or in combination with other enzymes at some point during the conditioning before milling. In one embodiment, the present invention provides compositions for the processing of cereals comprising a xylanase alone or in combination with other enzymes at some point during the conditioning before milling. In some embodiments, the other enzymes are other cell wall degrading enzymes. In some embodiments the invention provides compositions for the processing of cereals comprising g a beta glucanase/cellulase complex and/or a xylanase alone or in combination with other enzymes during the conditioning process.

In some embodiments, the compositions comprise an enzyme exhibiting xylanase activity, which enzyme comprises an amino acid sequence having at least 80% identity with any one selected from SEQ ID NO:1-SEQ ID NO:8, or any functional fragment thereof. In some embodiments, the compositions comprise an enzyme exhibiting xylanase activity, which enzyme comprises an amino acid sequence having at least 80% identity with any one selected from SEQ ID NO:1; SEQ ID NO:2, SEQ ID NO:7 and SEQ ID NO:8, or any functional fragment thereof. In some embodiments the enzyme composition according to the invention comprises a xylanase activity of at least about 5000 U/g, such as at least about 6000 U/g, such as at least about 7000 U/g, such as at least about 8000 U/g, such as at least about 8500 U/g, as measured by in the assays described herein or any suitable assay known in the art. In some embodiments, an enzyme exhibiting xylanase activity has a minimum activity of 13000 U/g. In some embodiments, the enzyme exhibiting xylanase activity is present in a liquid composition at an amount of 100000-300000 units per gram. In some embodiments, the one or more enzyme has at least 81, 82, 83, 84, 85, 86, 87, 88, 89, 90, 91, 92, 93, 94, 95, 96, 97, 98 or 99% identity with any one amino acid sequence described above.

In some embodiments, an enzyme composition comprising an enzyme exhibiting xylanase activity is added during the conditioning process at a concentration of 25-300 ppm, or 50-200 ppm, or 50-100 ppm, or 100-200 ppm, or 50-150 ppm. In some embodiments, an enzyme composition comprising an enzyme exhibiting xylanase is added during the conditioning process at a concentration of 50 ppm. In some embodiments, an enzyme composition comprising an enzyme exhibiting xylanase is added during the conditioning process at a concentration of 100 ppm. In some embodiments, an enzyme composition comprising an enzyme exhibiting xylanase is added during the conditioning process at a concentration of 150 ppm. In some embodiments, an enzyme composition comprising an enzyme exhibiting xylanase is added during the conditioning process at a concentration of 200 ppm. In some embodiments, the composition is a liquid composition. In some embodiments, the liquid composition comprising an enzyme exhibiting xylanase activity is combined with the conditioning water. In some embodiments, the conditioning process comprises spraying water containing the enzymes compositions described herein to the cereal grains. In some embodiments, an enzyme exhibiting xylanase activity has a minimum activity of 13000 U/g. In some embodiments, the enzyme exhibiting xylanase activity is present in a liquid composition at an amount of 100000-300000 units per gram.

In some embodiments the one or more cell-wall modifying enzyme(s) used in the methods or compositions according to the present invention is an enzyme complex containing a plurality of different enzyme activities having an amino acid sequence having at least 80% identity to the plurality of enzymes produced upon fermentation of Trichoderma reesei selected from the group of enzymes having GenBank accession no. consisting of M16190, M15665, M19373, AB003694, Y11113, Z33381, AY281371, AY281372, AY281373, U09580, AB003110, AY281374, AY281375, AY281377, AY281378, AY281379, X69574, X69573, AB036796, Z69257, Z69256, AY281376, Z69252, AY281369, L25310, Z69253, Z69254, Z69255, Z68706, AJ549427, AJ245918, AY281370, AY281368. In some embodiments, the compositions containing the enzyme complex comprising a plurality of enzymes has beta-glucanase activities of 1000-2000 U/g and cellulase activities of 6000-8000 U/g. In some embodiments, the one or more enzyme has at least 81, 82, 83, 84, 85, 86, 87, 88, 89, 90, 91, 92, 93, 94, 95, 96, 97, 98 or 99% identity with any one amino acid sequence described above.

In some embodiments, an enzyme composition comprising an enzyme complex exhibiting beta-glucanase and cellulase activities is added during the conditioning process at a concentration of 25-300 ppm, or 50-200 ppm, or 50-100 ppm, or 100-200 ppm, or 50-150 ppm. In some embodiments, an enzyme composition comprising an enzyme complex exhibiting beta-glucanase and cellulase activities is added during the conditioning process at a concentration of 50 ppm. In some embodiments, an enzyme composition comprising an enzyme complex exhibiting beta-glucanase and cellulase activities is added during the conditioning process at a concentration of 100 ppm. In some embodiments, an enzyme composition comprising an enzyme complex exhibiting beta-glucanase and cellulase activities is added during the conditioning process at a concentration of 150 ppm. In some embodiments, an enzyme composition comprising an enzyme complex exhibiting beta-glucanase and cellulase activities is added during the conditioning process at a concentration of 200 ppm. In some embodiments, the composition is a liquid composition. In some embodiments, the liquid composition comprising an enzyme complex exhibiting beta-glucanase and cellulase activities is combined with the conditioning water. In some embodiments, the conditioning process comprises spraying water containing the enzymes compositions described herein to the cereal grains. In some embodiments, the enzymes exhibiting beta-glucanase activities and the enzymes exhibiting cellulase activities are present in a liquid composition at an amount of 1000-2000 U/g, and 6000-8000 U/g, respectively. In some embodiments, the enzyme composition further comprise other enzymes are selected from the group consisting of arabinofuranosidase, xylosidase, mannanase, alpha-galactosidase, beta-glucuronidase, and beta-galactosidase.

In some embodiments the one or more cell-wall modifying enzyme used in the methods or compositions according to the present invention is an enzyme complex containing a plurality of different enzyme activities having an amino acid sequence having at least 80% identity to the plurality of enzymes produced upon fermentation of Trichoderma reesei selected from the group of enzymes having locus no. (from genome.jgi-psf.org/Trire2/Trire2.home.html) consisting of ORF_123283, ORF_76210, ORF_55319, ORF_54219, ORF_123989, ORF_123989, ORF_123989, ORF_123989, ORF_72567, ORF_72567, ORF_72567, ORF_72567, ORF_72567, ORF_72567, ORF_72567, ORF_72567, ORF_72567, ORF_72567, ORF_122081, ORF_120312, ORF_120312, ORF_123232, ORF_123232, ORF_49081, ORF_49081, ORF_49081, ORF_49081, ORF_27554, ORF_121127, ORF_121127, ORF_74223, ORF_123818, ORF_111849, ORF_56996, ORF_76672, and ORF_73897. In some embodiments, the compositions containing the enzyme complex comprising a plurality of enzymes has beta-glucanase activities of 1000-2000 U/g and cellulase activities of 6000-8000 U/g. In some embodiments, the one or more enzyme has at least 81, 82, 83, 84, 85, 86, 87, 88, 89, 90, 91, 92, 93, 94, 95, 96, 97, 98 or 99% identity with any one amino acid sequence described above.

In some embodiments, an enzyme composition comprising an enzyme complex exhibiting beta-glucanase and cellulase activities is added during the conditioning process at a concentration of 25-300 ppm, or 50-200 ppm, or 50-100 ppm, or 100-200 ppm, or 50-150 ppm. In some embodiments, an enzyme composition comprising an enzyme complex exhibiting beta-glucanase and cellulase activities is added during the conditioning process at a concentration of 50 ppm. In some embodiments, an enzyme composition comprising an enzyme complex exhibiting beta-glucanase and cellulase activities is added during the conditioning process at a concentration of 100 ppm. In some embodiments, an enzyme composition comprising an enzyme complex exhibiting beta-glucanase and cellulase activities is added during the conditioning process at a concentration of 150 ppm. In some embodiments, an enzyme composition comprising an enzyme complex exhibiting beta-glucanase and cellulase activities is added during the conditioning process at a concentration of 200 ppm. In some embodiments, the composition is a liquid composition. In some embodiments, the liquid composition comprising an enzyme complex exhibiting beta-glucanase and cellulase activities is combined with the conditioning water. In some embodiments, the conditioning process comprises spraying water containing the enzymes compositions described herein to the cereal grains. In some embodiments, the enzymes exhibiting beta-glucanase activities and the enzymes exhibiting cellulase activities are present in a liquid composition at an amount of 1000-2000 U/g, and 6000-8000 U/g, respectively. In some embodiments, the enzyme complex comprises other enzymes selected from the group consisting of arabinofuranosidase, xylosidase, mannanase, alpha-galactosidase, beta-glucuronidase, and beta-galactosidase.

The enzyme compositions used in the methods according to the present invention may be compositions produced by fermentation of selected bacterial strains, such as Trichoderma reesei or Bacillus subtilis. Enzyme compositions may contain water, stabilizing agents, such as sorbitol, and salts, such as sodium chloride, sodium benzoate, and potassium sorbate with a pH in the range of 4-6, such as 4.5-5. The enzyme compositions may be exempt from FDA labelling and approved for food products.

Preferred liquid enzymatic products are produced by using selected strains of bacteria and fungi. Preferred enzymatic products are liquid for easy handling. In some embodiments, the liquid compositions comprising the enzymes described herein easily mix and combine with the conditioning water. In some embodiments, the liquid compositions comprises the enzymes exhibiting beta-glucanase activities and the enzymes exhibiting cellulase activities at an amount of 1000-2000 U/g, and 6000-8000 U/g, respectively. In some embodiments, the liquid composition comprises other enzymes selected from the group consisting of arabinofuranosidase, xylosidase, mannanase, alpha-galactosidase, beta-glucuronidase, and beta-galactosidase.

In one aspect, the present invention provides compositions to increase the productivity and efficiency of flour mills. Enzyme compositions according to the present invention enable millers, e.g., to cut costs without compromising flour quality.

In some embodiments, the compositions according to the present invention provides for a wide range of different benefits in relation to the flour as well as the bran obtained from the methods and in relation to the bread obtained in e.g. baking application.

In some embodiments, the compositions described herein comprising one or more enzyme exhibiting cellulase activity and/or one or more enzymes exhibiting beta-glucanase activity, and/or one or more enzymes exhibiting xylanase activity, alone or in combination, provide for improved conditioning processes before milling. In some embodiments, the compositions described herein provide for alignment and shortening of conditioning time according to specific grain varieties, even when blending different grains varieties with different humidification needs.

In some embodiments, the compositions described herein comprising one or more enzyme exhibiting cellulase activity and/or one or more enzymes exhibiting beta-glucanase activity, and/or one or more enzymes exhibiting xylanase activity, alone or in combination, provide for opening of the wheat kernel structure during conditioning of the grain with water, which enables water to migrate faster between the different layers. This leads to shorter conditioning time, easier separation of the endosperm from the aleurone layer and results in a potentially higher extraction rate

In some embodiments, the compositions described herein comprising one or more enzyme exhibiting cellulase activity and/or one or more enzymes exhibiting beta-glucanase activity, and/or one or more enzymes exhibiting xylanase activity, alone or in combination, provide for reduce conditioning time (e.g. by 30-50%). In some embodiments, the compositions described herein reduce conditioning time by at least 10, 15, 20, 25, 30, 35, 40, 45, or 50%.

In some embodiments, the compositions described herein comprising one or more enzyme exhibiting cellulase activity and/or one or more enzymes exhibiting beta-glucanase activity, and/or one or more enzymes exhibiting xylanase activity, alone or in combination, provide for increase extraction rates (e.g., from 0.5%-2%). In some embodiments, the compositions described herein increase extraction rates, such as from at least about 0.5%, such as from at least about 0.6, 0.7, 0.8, 0.9, 1.0, 1.1, 1.2, 1.3, 1.4, 1.5, 1.6, 1.7, 1.8, 1.9, or 2, or even 5-10%.

In some embodiments, the compositions described herein comprising one or more enzyme exhibiting cellulase activity and/or one or more enzymes exhibiting beta-glucanase activity, and/or one or more enzymes exhibiting xylanase activity, alone or in combination, provide for energy savings (e.g. up to 20%, e.g. from 5% up to 20%). In some embodiments, the compositions described herein produce energy savings, such as up to 20%, such at up to 18, 16, 14, 12, 10, 8, 6, 5, 4, or 2%. In some embodiments, the compositions described herein produce energy savings, such as from about 5% up to about 20%.

In some embodiments, the compositions described herein comprising one or more enzyme exhibiting cellulase activity and/or one or more enzymes exhibiting beta-glucanase activity, and/or one or more enzymes exhibiting xylanase activity, alone or in combination, provide for whitening effect in the flour. In some embodiments, the compositions describe herein provide for shell/skin/crust (whole less broken) during conditioning.

In some embodiments, the compositions described herein comprising one or more enzyme exhibiting cellulase activity and/or one or more enzymes exhibiting beta-glucanase activity, and/or one or more enzymes exhibiting xylanase activity, alone or in combination, provide for stable flour quality.

Examples of other benefits provided by the compositions described herein, include but are not limited provides for easier separation of the bran, improvement of bran quality, lightener bread dough, when flour used in baking application, increased final bread volume when flour used in baking application, improved or not worsen ash level, improved color of flour with less impurities, no change to the flour quality or the baking performance, same high quality, reduced loss during the milling, increased whitening of the flour and/or more integral bran.

In some embodiments the compositions described herein for better dissolution of the liquid enzyme composition and less precipitation of enzyme in the conditioning water.

Accordingly, in some embodiments the enzyme composition according to the present invention reduce the conditioning time of a cereal grain such as wheat by at least 10, 15, 20, 25, 30, 35, 40, 45, or 50% as compared to a negative control cereal conditioned without said enzyme composition.

In some embodiments the enzyme composition according to the present invention increases extraction rates at least about 0.5%, such as from at least about 0.6, 0.7, 0.8, 0.9, 1.0, 1.1, 1.2, 1.3, 1.4, 1.5, 1.6, 1.7, 1.8, 1.9, or 2% compared to a negative control cereal conditioned without said enzyme composition.

In some embodiments the enzyme composition according to the present invention provide for energy savings up to 20%, such at up to 18, 16, 14, 12, 10, 8, 6, 5, 4, or 2% compared to a negative control cereal conditioned without said enzyme composition. In some embodiments, the compositions described herein produce energy savings, such as from about 5% up to about 20%.

In some embodiments, the composition comprising one or more cell-wall modifying enzyme(s) is a liquid, such as an aqua formulation.

In some embodiments, the composition comprising one or more cell-wall modifying enzyme(s) is an aqua formulation comprising the enzymes secreted from the fermentation of the genus Trichoderma, such as Trichoderma reesei, such as beta-glucanases or cellulases.

In some embodiments, the beta-glucanase and said cellulase is present in an amount of 10,000 to 1,000,000 AZO BBG U per ton cereal grain (75,000-340,000), and 100,000 to 10×10⁶ IU per ton cereal grain (310,000-1,516,000) respectively.

In some embodiments, the composition comprising one or more cell-wall modifying enzyme(s) is an aqua formulation comprising the enzymes secreted from the fermentation of the genus Bacillus, such as Bacillus subtilis, such as a bacterial xylanase.

In some embodiments, the bacterial xylanase is present in an amount of 1×10⁶ to 100×10⁶ units per ton cereal grain (9×10⁶ to 52×10⁶ in product sheet).

In some embodiments, the composition in step a) of the methods of the invention further comprises one or more oxidase.

Other aspects of the present invention relates to an aqua composition comprising an expression product obtained by fermentation of a species of the genus Trichoderma; which expression product comprises a beta-glucanase (EC 3.2.1.6) and a cellulase (EC 3.2.1.4), wherein said beta-glucanase is present in an amount of 1000-2000 AZO BBG U per gram aqua composition and said cellulase is present in an amount of 6000-8000 IU per gram aqua composition, or an aqua composition comprising an expression product obtained by fermentation of a species of the genus Bacillus; which expression product comprises a xylanase (EC 3.2.1.8), wherein said xylanase is present in an amount of 100000-300000 units per gram aqua composition.

In some embodiments, the aqua composition comprises an expression product obtained by fermentation of the genus Trichoderma is from the species Trichoderma reesei.

In some embodiments, the aqua composition comprises expression product obtained by fermentation of the genus Bacillus is from the species Bacillus subtilis.

Materials may be added to an enzyme-containing liquid to improve the properties of the liquid composition. Non-limiting examples of such additives include: salts (e.g., alkali salts, earth metal salts, additional chloride salts, sulfate salts, nitrate salts, carbonate salts, where exemplary counter ions are calcium, potassium, and sodium), inorganic minerals or clays (e.g., zeolites, kaolin, bentonite, talc's and/or silicates), carbohydrates (e.g., sucrose and/or starch), coloring pigments (e.g., titanium dioxide), biocides (e.g., Rodalon®, Proxel®), dispersants, anti-foaming agents, reducing agents, acid agents, alkaline agents, enzyme stabilizers (e.g. polyol such as glycerol, propylene glycol, sorbitol, inorganic salts, sugars, sugar or a sugar alcohol, lactic acid, boric acid, or a boric acid derivative and combinations thereof), enzyme inhibitors, preservative (e.g. methyl paraben, propyl paraben, benzoate, sorbate or other food approved preservatives) and combinations thereof. Excipients which may be used in the preparation/composition include maltose, sucrose, glucose including glucose syrup or dried glucose syrup, pre-cooked starch, gelatinized starch, L-lactic, ascorbyl palmitate, tocopherols, lecithins, citric acid, citrates, phosphoric, phosphates, sodium alginate, carrageenan, locust bean gum, guar gum, xanthan gum, pectins, sodium carboxymethylcellulose, mono- and diglycerides, citric acid esters of mono- and diglycerides, sucrose esters, carbon dioxide, argon, helium, nitrogen, nitrous oxide, oxygen, hydrogen, and starch sodium octenylsuccinate.

### Methods

In one aspect, the present disclosure provides methods for cereal processing, e.g., wheat conditioning. In some embodiments, the present invention relates to methods of processing of cereal grains, in particular wheat, for milling as well as optimized enzyme compositions suitable for that particular purpose. In some embodiments, the present invention provides methods for improved processing of cereals, e.g., reduced conditioning time, reduce energy consumption and/or increase extraction rate. In some embodiments, the present invention provides methods for improved conditioning of cereals which improve the milling processes.

In some embodiments, the present invention provides methods for the conditioning of cereals comprising adding water to the cereal grain, conditioning the cereal grain for a specific amount of time for the cereal grain to absorb the water and one or more enzymes as described herein which are added one or more times during the process. In some embodiments, the present invention is directed to methods for the processing of cereals comprising adding one or more enzymes to the conditioning step of the cereal grains before milling. In some embodiments, the present invention provides methods for the processing of cereals comprising adding a cell wall degrading enzyme alone or in combination with other enzymes at some point during the conditioning before milling. In some embodiments, the cell wall degrading enzyme is a xylanase or a beta glucanase. In some embodiments, the cell wall degrading enzyme is a xylanase and/or a beta glucanase complex. In one embodiment, the present invention provides methods for the processing of cereals comprising adding one or more beta glucanases alone, and/or one or more cellulases alone, and/or in combination with other enzymes at some point during the conditioning before milling. In one embodiment, the present invention provides methods for the processing of cereals comprising adding a xylanase alone or in combination with other enzymes at some point during the conditioning before milling. In some embodiments, the other enzymes are other cell wall degrading enzymes. In some embodiments the invention provides methods for the processing of cereals comprising adding a beta glucanase/cellulase complex and/or a xylanase alone or in combination with other enzymes during the conditioning process. In some embodiments, the other enzymes are selected from the group consisting of arabinofuranosidase, xylosidase, mannanase, alpha-galactosidase, beta-glucuronidase, and beta-galactosidase.

In the conditioning method according to the present invention water is added to grains, such as wheat, in the presence of one or more enzymes described herein. Water may be added prior to or in conjunction to the addition of the liquid composition comprising one or more cell-wall modifying enzyme. Accordingly, water may be added first to allow some conditioning or tempering of the grains before the enzyme composition is added or the enzyme composition may be added simultaneously with the conditioning water. In some embodiments enzyme composition is added by a system that may be set or that adjust the amount of enzyme being added to the grain. The volume of water per ton of grain, time of conditioning with or without enzyme may be independently varied depending on specific type of grain, milling process, or the subsequent use of the conditioned grain.

In the Examples described herein it has surprisingly been found that adding a liquid composition comprising the one or more one or more cell-wall modifying enzymes into the conditioning water provides for improved processing of cereals, e.g., reduced conditioning time, reduce energy consumption and/or increase extraction rate. Thus, in some embodiments, water containing the liquid composition comprising one or more cell-wall modifying enzymes is added to the cereal grain (e.g. wheat). In some embodiments, a dosing system is used to combine the liquid composition comprising one or more cell-wall modifying enzymes and the conditioning water. In some embodiments, a mixing system (e.g. an agitator) is used to mix and maintain homogenous mixing of the enzymes in the conditioned water.

Further, it was surprisingly found that spraying conditioning water containing the enzymes described herein onto the cereal grains provides for improved processing of cereals, e.g., reduced conditioning time, reduce energy consumption and/or increase extraction rate. Thus, in some embodiments, water containing the liquid composition comprising one or more cell-wall modifying enzymes is sprayed to the cereal grain (e.g. wheat). In some embodiments, the conditioning step involve spraying the cereal, and one or more enzymes as described herein are added one or more times during the process. In some embodiments, the one or more enzymes as described herein are added constantly during the spraying of the cereal. In some embodiments, a dosing system is used to combine the liquid composition comprising one or more cell-wall modifying enzymes and the conditioning water. In some embodiments, a mixing system (e.g. an agitator) is used to mix and maintain homogenous mixing of the enzymes in the conditioned water.

In some embodiments, an enzyme composition comprising one or more cell-wall modifying enzymes is added during the conditioning process at a concentration of 25-300 ppm, or 50-200 ppm, or 50-100 ppm, or 100-200 ppm, or 50-150 ppm. In some embodiments, an enzyme composition comprising one or more cell-wall modifying enzymes is added during the conditioning process at a concentration of 50 ppm. In some embodiments, an enzyme composition comprising one or more cell-wall modifying enzymes is added during the conditioning process at a concentration of 100 ppm. In some embodiments, an enzyme composition comprising one or more cell-wall modifying enzymes is added during the conditioning process at a concentration of 150 ppm. In some embodiments, an enzyme composition comprising one or more cell-wall modifying enzymes is added during the conditioning process at a concentration of 200 ppm. In some embodiments, the composition is a liquid composition. In some embodiments, the one or more cell-wall modifying enzymes are an enzyme exhibiting xylanase activity having a minimum activity of 13000 U/g. In some embodiments, the one or more cell-wall modifying enzymes are enzyme exhibiting xylanase activity present in the liquid composition at an amount of 100000-300000 units per gram. In some embodiments, the one or more cell-wall modifying enzymes are enzymes exhibiting beta-glucanase activities and/or enzymes exhibiting cellulase activities present in a liquid composition at an amount of 1000-2000 U/g, and 6000-8000 U/g, respectively. In some embodiments, the enzyme composition further comprises other enzymes selected from the group consisting of arabinofuranosidase, xylosidase, mannanase, alpha-galactosidase, beta-glucuronidase, and beta-galactosidase.

Thus, in some embodiments, the conditioning step involve spraying the cereal with conditioning water containing an enzyme composition comprising one or more cell-wall modifying enzymes, wherein the enzyme composition described herein is added during the conditioning process at a concentration of 25-300 ppm, or 50-200 ppm, or 50-100 ppm, or 100-200 ppm, or 50-150 ppm. In some embodiments, the enzyme composition as described herein is added constantly during the spraying of the cereal. In some embodiments, a dosing system is used to combine the liquid composition comprising one or more cell-wall modifying enzymes and the conditioning water. In some embodiments, the enzyme composition comprises one or more cell-wall modifying enzymes exhibiting xylanase activity having a minimum activity of 13000 U/g. In some embodiments, the enzyme composition comprises enzyme exhibiting xylanase activity present in the liquid composition at an amount of 100000-300000 units per gram. In some embodiments, the enzyme composition comprises enzymes exhibiting beta-glucanase activities and/or enzymes exhibiting cellulase activities present in a liquid composition at an amount of 1000-2000 U/g, and 6000-8000 U/g, respectively. In some embodiments, the enzyme composition further comprises other enzymes selected from the group consisting of arabinofuranosidase, xylosidase, mannanase, alpha-galactosidase, beta-glucuronidase, and beta-galactosidase.

The concentration of one or more enzymes described herein (e.g., a beta glucanase and/or a xylanase) may vary depending on the conditions of the conditioning process, the type of cereal (e.g., β-glucans and arabinoxylans content in the grain), the desired specification of the final product (e.g. stickiness of flour, β-glucans and arabinoxylans content and moisture level of the milled product, etc. . . . ) and the type of enzyme(s) being utilized. In some embodiments, the conditioning process will vary depending on the actual type of grain and in particular whether it is a soft, mid hard or hard grain. A “soft grain” denotes a grain with the following average characteristics: W=80-150, P/L=0.2-0.5 as measured on an Alveograph (W: strength; P: 15 Tenacity; L: extensibility); a “mid hard grain” or “middle hard grain” denotes a grain with the following average characteristics W=150-300, P/L=0.5-0.8 as measured on an Alveograph; and a “hard grain” denotes a grain with the following average characteristics: W=300-20400, P/L=0.8-1 as measured on an Alveograph. The grain hardness can also be determined by the Near Infra-Red method (NIR). NIR is typically used in the wheat mills to measure the wheat hardness. The hardness of wheat using NIR is classified as follows: less than 45% as “soft grain”; 45%-60% as “mid hard grain” or “middle hard grain”; and higher than 60% as “hard grain”. Other methods such Brabender or Perkens that measure particle size can also be used to determine grain hardness.

In some embodiments, the methods comprise an enzyme having xylanase activity, which enzyme comprises an amino acid sequence having at least 80% identity with any one selected from SEQ ID NO:1-SEQ ID NO:8, or any functional fragment thereof. In some embodiments, the methods comprise an enzyme exhibiting xylanase activity, which enzyme comprises an amino acid sequence having at least 80% identity with any one selected from SEQ ID NO:1; SEQ ID NO:2, SEQ ID NO:7 and SEQ ID NO:8, or any functional fragment thereof. In some embodiments, the methods comprise an enzyme having xylanase activity, which enzyme comprises an amino acid sequence having at least 90% identity with any one selected from SEQ ID NO:1-SEQ ID NO:8, or any functional fragment thereof. In some embodiments, the methods comprise an enzyme exhibiting xylanase activity, which enzyme comprises an amino acid sequence having at least 90% identity with any one selected from SEQ ID NO:1; SEQ ID NO:2, SEQ ID NO:7 and SEQ ID NO:8, or any functional fragment thereof, or any functional fragment thereof. In some embodiments, the methods comprise an enzyme having xylanase activity, which enzyme comprises an amino acid sequence having at least 95% identity with any one selected from SEQ ID NO:1-SEQ ID NO:8, or any functional fragment thereof. In some embodiments, the methods comprise an enzyme exhibiting xylanase activity, which enzyme comprises an amino acid sequence having at least 95% identity with any one selected from SEQ ID NO:1; SEQ ID NO:2, SEQ ID NO:7 and SEQ ID NO:8, or any functional fragment thereof, or any functional fragment thereof.

In some embodiments, the methods comprise an enzyme exhibiting beta-glucanase and/or cellulase and/or other enzymes activity selected from the group consisting of an enzyme complex containing a plurality of different enzyme activities having an amino acid sequence having at least 80% identity to the plurality of enzymes produced upon fermentation of Trichoderma reesei selected from the group of enzymes having GenBank accession no. consisting of M16190, M15665, M19373, AB003694, Y11113, Z33381, AY281371, AY281372, AY281373, U09580, AB003110, AY281374, AY281375, AY281377, AY281378, AY281379, X69574, X69573, AB036796, Z69257, Z69256, AY281376, Z69252, AY281369, L25310, Z69253, Z69254, Z69255, Z68706, AJ549427, AJ245918, AY281370, AY281368, or any functional fragment thereof. In some embodiments, the methods comprise an enzyme exhibiting beta-glucanase and/or cellulase and/or other enzymes activity selected from the group consisting of an enzyme complex containing a plurality of different enzyme activities having an amino acid sequence having at least 80% identity to the plurality of enzymes produced upon fermentation of Trichoderma reesei selected from the group of enzymes having locus no. (from genome.jgi-psf.org/Trire2/Trire2.home.html) consisting of ORF_123283, ORF_76210, ORF_55319, ORF_54219, ORF_123989, ORF_123989, ORF_123989, ORF_123989, ORF_72567, ORF_72567, ORF_72567, ORF_72567, ORF_72567, ORF_72567, ORF_72567, ORF_72567, ORF_72567, ORF_72567, ORF_122081, ORF_120312, ORF_120312, ORF_123232, ORF_123232, ORF_49081, ORF_49081, ORF_49081, ORF_49081, ORF_27554, ORF_121127, ORF_121127, ORF_74223, ORF_123818, ORF_111849, ORF_56996, ORF_76672, and ORF_73897. In some embodiments, the methods comprise an enzyme exhibiting beta-glucanase activity in combination with an enzyme exhibiting xylanase activity, each independently selected from the groups described above. In some embodiments, the one or more enzyme has at least 81, 82, 83, 84, 85, 86, 87, 88, 89, 90, 91, 92, 93, 94, 95, 96, 97, 98 or 99% identity with any one amino acid sequence described above.

In some embodiments the methods according to the invention comprises the use of any suitable commercially available enzymes having xylanase activity and/or beta-glucanase activity such as UltraFlo L (available from Novozymes—beta glucanase with cellulase, xylanase side activities), UltraFlo XL (available from Novozymes—beta glucanase with xylanase and alpha amylase side activities), UltraFlo Max (available from Novozymes—beta glucanase and xylanase), Finizyme 250 L (available from Novozymes—beta glucanase with cellulase, xylanase side activities) Filtrase series (available from DSM-beta glucanase and xylanases).

In some embodiments, the methods described herein provide for alignment and shortening of conditioning time according to specific grain varieties, even when blending different grains varieties with different humidification needs.

In some embodiments, the present invention provides methods for the conditioning of a hard cereal grain. In some embodiments, an enzyme composition comprising one or more cell-wall modifying enzymes is added during the conditioning process of a hard grain at a concentration of 50-200 ppm, for about 6 to 12 hours. In some embodiments, an enzyme composition comprising one or more cell-wall modifying enzymes is added during the conditioning process of a hard grain until the grain reaches a moisture of about 15% to about 17%, e.g. 15.5% to 17%. In some embodiments, an enzyme composition comprising one or more cell-wall modifying enzymes is added during the conditioning process of a hard grain until the grain goes from a moisture of about 10.5%-14% to a moisture of about 15% to about 17%, e.g. 15.5% to 17%. In some embodiments, the hard grain is Durum. In some embodiments, an enzyme composition comprising one or more cell-wall modifying enzymes is added during the conditioning process of a Durum grain until the grain reaches a moisture of about 14% to about 16%, e.g. 14% to 15.5%. In some embodiments, an enzyme composition comprising one or more cell-wall modifying enzymes is added during the conditioning process of a Durum grain until the grain goes from a moisture of about 11.5%-13.8% to a moisture of about 14% to about 16%, e.g. 14% to 5.5%. In some embodiments, the hard grain is Durum.

In some embodiments, the present invention provides methods for the conditioning of a mid-hard cereal grain. In some embodiments, an enzyme composition comprising one or more cell-wall modifying enzymes is added during the conditioning process of a mid-hard grain at a concentration of 50-200 ppm, for about 6 to 12 hours.

In some embodiments, the present invention provides methods for the conditioning of a soft cereal grain. In some embodiments, an enzyme composition comprising one or more cell-wall modifying enzymes is added during the conditioning process of a soft grain at a concentration of 50-150 ppm, for about 6 to 12 hours. In some embodiments, an enzyme composition comprising one or more cell-wall modifying enzymes is added during the conditioning process of a soft grain at a concentration of 50-150 ppm, for about 6 or less. In some embodiments, an enzyme composition comprising one or more cell-wall modifying enzymes is added during the conditioning process of a soft grain until the grain reaches a moisture of about 15% to about 17%, e.g. 15%-16.5%. In some embodiments, an enzyme composition comprising one or more cell-wall modifying enzymes is added during the conditioning process of a hard grain until the grain goes from a moisture of about 12.5%-14% to a moisture of about 15% to about 17%, e.g. 15%-16.5%.

In some embodiments, the cereal and one or more enzymes described herein (e.g., a beta glucanase and/or a xylanase) are combined at an effective concentration during the wetting of the cereal, and the combination is held at a temperature (e.g. of at least about 5° C., about 30° C., or about 10° C. to about 20° C.) for a period of time to hydrolyze at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the β-glucans and/or arabinoxylans in grain. In some embodiments, the cereal and one or more enzymes described herein (e.g., a beta glucanase and/or a xylanase) are combined to hydrolyze at least 50% of the β-glucans and/or arabinoxylans in grain. In some embodiments, the cereal and one or more enzymes described herein (e.g., a beta glucanase and/or a xylanase) are combined to hydrolyze at least 60% of the β-glucans and/or arabinoxylans in grain. In some embodiments, the cereal and one or more enzymes described herein (e.g., a beta glucanase and/or a xylanase) are combined to hydrolyze at least 70% of the β-glucans and/or arabinoxylans in grain. In some embodiments, the cereal and one or more enzymes described herein (e.g., a beta glucanase and/or a xylanase) are combined to hydrolyze at least 80% of the β-glucans and/or arabinoxylans in grain. In some embodiments, the cereal and one or more enzymes described herein (e.g., a beta glucanase and/or a xylanase) are combined to hydrolyze at least 50% of the β-glucans and/or arabinoxylans in grain but not more than 80%. In some embodiments, the cereal and one or more enzymes described herein (e.g., a beta glucanase and/or a xylanase) are combined to hydrolyze at least 60% of the β-glucans and/or arabinoxylans in grain but not more than 80%. In some embodiments, the cereal and one or more enzymes described herein (e.g., a beta glucanase and/or a xylanase) are combined to hydrolyze at least 70% of the β-glucans and/or arabinoxylans in grain but not more than 80%. In some embodiments, the cereal and one or more enzymes described herein (e.g., a beta glucanase and/or a xylanase) are combined to hydrolyze 80% of the β-glucans and/or arabinoxylans in grain.

In some embodiments, at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the β-glucans and/or arabinoxylans in grain are broken down compared to a control cereal prepared without the one or more enzymes according to the methods described herein. In some embodiments, at least 10% of the β-glucans and/or arabinoxylans in grain are broken down compared to a control cereal prepared without the one or more enzymes according to the methods described herein. In some embodiments, at least 50% of the β-glucans and/or arabinoxylans in grain are broken down compared to a control cereal prepared without the one or more enzymes according to the methods described herein. In some embodiments, at least 60% of the β-glucans and/or arabinoxylans in grain are broken down compared to a control cereal prepared without the one or more enzymes according to the methods described herein. In some embodiments, at least 70% of the β-glucans and/or arabinoxylans in grain are broken down compared to a control cereal prepared without the one or more enzymes according to the methods described herein. In some embodiments, at least 80% of the β-glucans and/or arabinoxylans in grain are broken down compared to a control cereal prepared without the one or more enzymes according to the methods described herein. In some embodiments, at least 10%, but not more than 80%, of the β-glucans and/or arabinoxylans in grain are broken down compared to a control cereal prepared without the one or more enzymes according to the methods described herein. In some embodiments, at least 50%, but not more than 80%, of the β-glucans and/or arabinoxylans in grain are broken down compared to a control cereal prepared without the one or more enzymes according to the methods described herein. In some embodiments, at least 60%, but not more than 80%, of the β-glucans and/or arabinoxylans in grain are broken down compared to a control cereal prepared without the one or more enzymes according to the methods described herein. In some embodiments, at least 70%, but not more that 80%, of the β-glucans and/or arabinoxylans in grain are broken down compared to a control cereal prepared without the one or more enzymes according to the methods described herein. In some embodiments, 80% of the β-glucans and/or arabinoxylans in grain are broken down compared to a control cereal prepared without the one or more enzymes according to the methods described herein.

In some embodiments, at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the β-glucans in grain are broken down compared to a control cereal prepared without the one or more enzymes according to the methods described herein. In some embodiments, at least 10% of the β-glucans in grain are broken down compared to a control cereal prepared without the one or more enzymes according to the methods described herein. In some embodiments, at least 50% of the β-glucans in grain are broken down compared to a control cereal prepared without the one or more enzymes according to the methods described herein. In some embodiments, at least 60% of the β-glucans in grain are broken down compared to a control cereal prepared without the one or more enzymes according to the methods described herein. In some embodiments, at least 80% of the β-glucans in grain are broken down compared to a control cereal prepared without the one or more enzymes according to the methods described herein. In some embodiments, at least 10%, but not more than 80%, of the β-glucans in grain are broken down compared to a control cereal prepared without the one or more enzymes according to the methods described herein. In some embodiments, at least 50%, but not more than 80%, of the β-glucans in grain are broken down compared to a control cereal prepared without the one or more enzymes according to the methods described herein. In some embodiments, at least 60%, but not more than 80%, of the β-glucans in grain are broken down compared to a control cereal prepared without the one or more enzymes according to the methods described herein. In some embodiments, 80% of the β-glucans in grain are broken down compared to a control cereal prepared without the one or more enzymes according to the methods described herein.

In some embodiments, the β-glucans in grain are broken down such that the concentration of high molecular weight β-glucan in the conditioned cereal is less than 200 mg/l, or 150 mg/l, or less than 100 mg/l or less than 90 mg/l, or less than 80 mg/l, or less than 70 mg/l, or less than 60 mg/l, or less than 50 mg/l. In some embodiments, the β-glucans in grain are broken down such that the concentration of high molecular weight β-glucan in the conditioned cereal is 50 mg/l or less. In some embodiments, the β-glucans in grain are broken down such that the concentration of high molecular weight β-glucan in the conditioned cereal is no less than 150 mg/l. In some embodiments, the β-glucans in grain are broken down such that the concentration of high molecular weight β-glucan in the conditioned cereal is no less than 50 mg/l.

In some embodiments, at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the arabinoxylans in grain are broken down compared to a control cereal prepared without the one or more enzymes according to the methods described herein. In some embodiments, at least 10% of the arabinoxylans in grain are broken down compared to a control cereal prepared without the one or more enzymes according to the methods described herein. In some embodiments, at least 10%, but no more than 80%, of the arabinoxylans in grain are broken down compared to a control cereal prepared without the one or more enzymes according to the methods described herein.

In some embodiments, at least 50% of the β-glucans in grain are broken down and at least 50% of the arabinoxylans are broken down compared to a control cereal prepared without the one or more enzymes according to the methods described herein. In some embodiments, at least 60% of the β-glucans in grain are broken down and at least 50% of the arabinoxylans are broken down compared to a control cereal prepared without the one or more enzymes according to the methods described herein. In some embodiments, at least 70% of the β-glucans in grain are broken down and at least 50% of the arabinoxylans are broken down compared to a control cereal prepared without the one or more enzymes according to the methods described herein. In some embodiments, at least 80% of the β-glucans in grain are broken down and at least 50% of the arabinoxylans are broken down compared to a control cereal prepared without the one or more enzymes according to the methods described herein. In some embodiments, at least 50%, but not more than 80%, of the β-glucans in grain are broken down and at least 50%, but not more than 80%, of the arabinoxylans are broken down compared to a control cereal prepared without the one or more enzymes according to the methods described herein. In some embodiments, at least 60%, but not more than 80%, of the β-glucans in grain are broken down and at least 50%, but not more than 80%, of the arabinoxylans are broken down compared to a control cereal prepared without the one or more enzymes according to the methods described herein. In some embodiments, at least 70%, but not more than 80%, of the β-glucans in grain are broken down and at least 50%, but not more than 80%, of the arabinoxylans are broken down compared to a control cereal prepared without the one or more enzymes according to the methods described herein. In some embodiments, 80% of the β-glucans in grain are broken down and at least 50%, but not more than 80%, of the arabinoxylans are broken down compared to a control cereal prepared without the one or more enzymes according to the methods described herein.

In some embodiments, the arabinoxylans in grain are broken down such that the concentration of high molecular weight arabinoxylans in the cereal is less than less than 2400 mg/l, or less than 2200 mg/l, or less than 1900 mg/l, or less 1500 than mg/l, or less than 1000 mg/l, or less than 800 mg/l, or less than 700 mg/l or less than 600 mg/l, or less than 500 mg/l, or less than 100 mg/l, or less than 60 mg/l, or less than 50 mg/l. In some embodiments, the arabinoxylans in grain are broken down such that the concentration of high molecular weight arabinoxylans in the conditioned cereal is 2000 mg/l or less. In some embodiments, the arabinoxylans in grain are broken down such that the concentration of high molecular weight arabinoxylans in the conditioned cereal is no less than 2000 mg/l. In some embodiments, the arabinoxylans in grain are broken down such that the concentration of high molecular weight arabinoxylans in the conditioned cereal is no less than 1000 mg/l.

In some embodiments, at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% of the cellulose in grain is broken down compared to a control cereal prepared without the one or more enzymes according to the methods described herein. In some embodiments, at least 10% of the cellulose in grain is broken down compared to a control cereal prepared without the one or more enzymes according to the methods described herein.

In some embodiments, the cereal and one or more enzymes described herein (e.g., a beta glucanase and/or a xylanase) are combined at an effective concentration during the wetting of the cereal at a specific temperature(s) for a period of time until the cereal has a moisture content of at least about 10 to 30% weight percent, or about 10 to 20%, or about 12 to 17%.

In still other embodiments, the cereal and the one or more enzymes are combined at an effective concentration during the wetting of the cereal for a period of time and temperature until the cereal has a moisture content of between about 10 to about 30 weight percent, or about 10 to 20 weight percent, and the β-glucans in grain are broken down such that the concentration of high molecular weight β-glucan in the cereal is 150 mg/l or less. In some embodiments the cereal and the one or more enzymes are combined at an effective concentration during the wetting of the cereal for a period of time and temperature until the cereal has a moisture content of between about 12 to about 17 weight percent, and the β-glucans in grain are broken down such that the concentration of high molecular weight β-glucan in the cereal is 150 mg/l or less. In still other embodiments, the cereal and the one or more enzymes are combined at an effective concentration during the wetting of the cereal for a period of time and temperature until the cereal has a moisture content of between about 10 to about 30 weight percent, or about 10 to 20 weight percent, and the β-glucans in grain are broken down such that the concentration of high molecular weight β-glucan in the cereal is no less than 50 mg/l. In some embodiments the cereal and the one or more enzymes are combined at an effective concentration during the wetting of the cereal for a period of time and temperature until the cereal has a moisture content of between about 12 to about 17 weight percent, and the β-glucans in grain are broken down such that the concentration of high molecular weight β-glucan in the cereal is no less than 50 mg/l. In some embodiments, the total condition time is less compared to the time of a control cereal prepared without the one or more enzymes according to the methods described herein.

In still other embodiments, the cereal and the one or more enzymes are combined at an effective concentration during one or more of the wetting of the cereal for a period of time and temperature until the cereal has a moisture content of between about 10 to about 30 weight percent, or about 10 to 20 weight percent, and the arabinoxylans in grain are broken down such that the concentration of high molecular weight arabinoxylans in the cereal is 1000 mg/l or less. In some embodiments the moistened cereal and the one or more enzymes are combined at an effective concentration during one or more of the wetting of the cereal for a period of time and temperature until the cereal has a moisture content of between about 12 to about 17 weight percent, and the arabinoxylans in grain are broken down such that the concentration of high molecular weight arabinoxylans in the cereal is 1000 mg/l or less. In still other embodiments, the cereal and the one or more enzymes are combined at an effective concentration during one or more of the wetting of the cereal for a period of time and temperature until the cereal has a moisture content of between about 10 to about 30 weight percent, or about 10 to 20 weight percent, and the arabinoxylans in grain are broken down such that the concentration of high molecular weight arabinoxylans in the cereal is no less 500 mg/l. In some embodiments the moistened cereal and the one or more enzymes are combined at an effective concentration during one or more of the wetting of the cereal for a period of time and temperature until the cereal has a moisture content of between about 12 to about 17 weight percent, and the arabinoxylans in grain are broken down such that the concentration of high molecular weight arabinoxylans in the cereal is no less than 500 mg/l. In some embodiments, the total conditioning time is less compared to the time of a control cereal prepared without the one or more enzymes according to the methods described herein.

In some embodiments, the total conditioning time is at least 5, 7, 9, 10, 11, 13, 15, 17, 19, 20, 21, 23, 25, 27, 29, 30, 31, 33, 35, 37, 39, 40, 41, 43, 45, 47, 49, 50, 51, 53, 55, 57, 59, 60, 61, 63, 65, 67, 69, 70, 71, 73, 75, 77, 79, 80, 81, 83, 85, 87, 89, 91, 93 or 95% less than the time of a control cereal prepared without the one or more enzymes according to the methods described herein.

In some embodiments, the total conditioning time does not exceed 12 hours. In some embodiments, the total conditioning time does not exceed 11 hours. In some embodiments, the total conditioning time is about 6 hours to about 12 hours. In some embodiments, the total conditioning time is less than 15, 12, 10, 8 or even less than 6 hours.

In some embodiments, the invention provides for improved water uptake by the grain. In some embodiments, the water uptake by the grain is improved by at least 10, 15, 20, 50, 70, 90 or 95% compared to a control cereal prepared without the one or more enzymes according to the methods described herein. In some embodiments, the water uptake by the grain is improved by at least 10%, or at least 20%.

In some embodiments, the cereal and one or more enzymes described herein (e.g., a beta glucanase, cellulase and/or a xylanase) are combined at an effective concentration at the beginning of the wetting of the cereal. In some embodiments, the cereal and one or more enzymes described herein (e.g., a beta glucanase, cellulase and/or a xylanase) are combined at an effective concentration during the wetting of the cereal. In some embodiments, the cereal and one or more enzymes described herein (e.g., a beta glucanase, cellulase and/or a xylanase) are combined at an effective concentration during the wetting of the cereal after a desired temperature have been reached. In some embodiments, the wetting stage involves spraying the cereal, where one or more enzymes as described herein are added one or more times, or constantly, during the spraying.

In some embodiments, the cereal and one or more enzymes described herein (e.g., a beta glucanase, cellulase and/or a xylanase) are combined at an effective concentration 1, 2, 3 or 4 hours after the beginning of the wetting of the cereal. In some embodiments, the cereal and one or more enzymes described herein (e.g., a beta glucanase and/or a xylanase) are combined during the last hour of the wetting of the cereal.

In some embodiments, the present invention provides cereals, obtained according to the process of the invention, which present improved properties. For instance, the cereals produced by the methods described herein have a lower high molecular weight fraction of beta-glucan, and/or arabinoxylans, and/or cellulose content. This allows for a better processability of the cereal, e.g., in milling. In some embodiments, the cereals produced by the methods described herein have a concentration of high molecular weight β-glucan of 200 mg/ml, 100 mg/l, 90 mg/l, 80 mg/l, 70 mg/l, 60 mg/l, or 50 mg/l. In some embodiments, the cereals produced by the methods described herein have a concentration of high molecular weight arabinoxylans of 1000 mg/ml, 800 mg/l, 700 mg/l, 600 mg/l, 500 mg/l, 100 mg/l, or 50 mg/l.

In some embodiments, the methods describes herein provide for a cereal with a decrease amount of high molecular weight fraction of β-glucans and/or arabinoxylans after conditioning prior to milling. In some embodiments, the total amount of high molecular weight β-glucans and/or arabinoxylans is at least 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93 or 95% less than the amount of high molecular weight β-glucans and/or arabinoxylans in a control cereal prepared without the one or more enzymes according to the methods described herein.

In some embodiments, the methods describes herein provide for a cereal with wet gluten of at least 20, 21, 22, 23, 24, 25, 26, 27, 28, 29, 30, 31, 32, 33, 34, or 35%. In some embodiments, the methods describes herein provide for a cereal with wet gluten of at least 24%. In some embodiments, the methods describes herein provide for a cereal with wet gluten of at least 29%. In some embodiments, the methods describes herein provide for a cereal with wet gluten of at least 30%. In some embodiments, the methods describes herein provide for a cereal with wet gluten of at least 31%.

In some embodiments, the methods describes herein provide for a cereal with moisture of at least about 12, 13, 14, 15, 16, 17, 18, 19 or 20%. In some embodiments, the methods describes herein provide for a cereal with moisture of at least about 13%. In some embodiments, the methods describes herein provide for a cereal with moisture of at least about 14%. In some embodiments, the methods describes herein provide for a cereal with moisture of at least about 15%. In some embodiments, the methods describes herein provide for a cereal with moisture of at least about 16%. In some embodiments, the methods describes herein provide for a cereal with moisture of at least about 17%.

In some embodiments the methods according to the present invention increases extraction rates at least about 0.5%, such as from at least about 0.6, 0.7, 0.8, 0.9, 1.0, 1.1, 1.2, 1.3, 1.4, 1.5, 1.6, 1.7, 1.8, 1.9, or 2% compared to a negative control cereal conditioned without said enzyme composition.

As mentioned above in one aspect the present invention relates to methods for the conditioning of a cereal grain, the method comprising the steps of: a) adding water in combination with a liquid composition comprising one or more cell-wall modifying enzyme(s) to the cereal grain; and b) conditioning the cereal grain for a specific amount of time for the cereal grain to absorb the water in the presence of said one or more cell-wall modifying enzyme(s).

In some embodiments, the cereal grain is wheat.

In some embodiments, the one or more cell-wall modifying enzyme(s) is selected from the group consisting of a xylanase, and a cellulase, such as cellobiohydrolases, endo-glucanases, and beta-glucanase. In some embodiments, the one or more cell-wall modifying enzyme(s) further comprises other enzymes selected from the group consisting of arabinofuranosidase, xylosidase, mannanase, alpha-galactosidase, beta-glucuronidase, and beta-galactosidase.

In some embodiments, the conditioning is performed for more than 6 hours, such as for more than 8, 10, 12, 14, 16, 18, 20, 22, 24, 26, 28, 30, 32, 34, 36, 38, or 40 hours.

In some embodiments, the conditioning is performed for less than 40 hours, such as for less than 38, 36, 34, 32, 30, 28, 26, 24, 22, 20, 18, 16, 14, 12, 10, or 8 hours.

The processes of conditioning cereals and milling are well known in the art and the specific conditions vary depending on the type of cereal used, the final specifications needed for the conditions cereals, the flour and the specific milling facilities. The methods described herein can be applied to any conditioning and milling process known in the art and can be used in any milling facility. It is to be understood that certain parameters of the compositions and methods described herein may be adjusted depending to the properties needed for the flour and/or the specific milling facilities to obtain such optimal properties in the conditioned cereal and flour, e.g., depending on the cereal varieties and the downstream uses

### EXAMPLES

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### Example 1

In this Example two different enzymes compositions were used: (i) an enzyme composition hydrolyzing beta-glucans and non-starch polysaccharides like arabinoxylans produced by fermentation of Trichoderma reesei having beta-glucanase activity of 1500-1700 AZO BBG U/g and cellulase activity of 6200-7580 IU/g (referred herein as “Cellulase/Beta-glucanase complex”); and (ii) bacterial xylanase having activity of 180000 units/g.

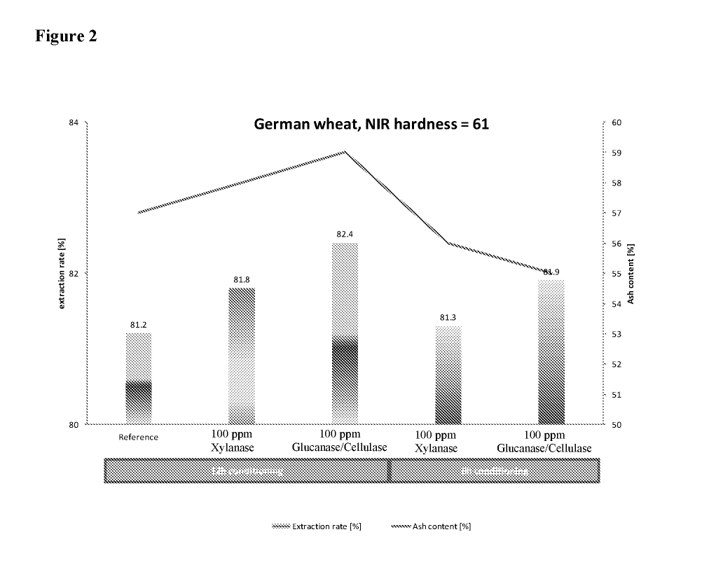

Trials with the wheat variety KERUBINO resulted in a significant higher extraction rate by reduced conditioning time. The results are demonstrated in FIG. 2

It is important that flour quality does not change significantly when the extraction rate is increased so that flour quality remains the same. Sometimes slightly higher ash content can be measured, but has no impact on baking performance or flour color as shown in Table 2. Results were confirmed in several trials and Table 1 illustrates results with American hard wheat.

TABLE 1
Conditioning time reduced up to
60% and increased extraction rate. American
hard wheat.

| | | Reduced conditioning time | |
|---|---|---|---|
| | | 100 ppm | |
| | | Cellulase/Beta- | |
| | | glucanase | 100 ppm |
| | Reference | complex | Xylanase |
| Conditioning time [hours] | 18 | 7 | 7 |
| Extraction measured [%] | 76.4 | 77.2 | 77.0 |
| Flour flow [kg/h] | 16100 | 16100 | 16100 |
| Rolls engine power [A] | Reference | Around 10% | Around 10% |
| | | reduction | reduction |

Energy Savings

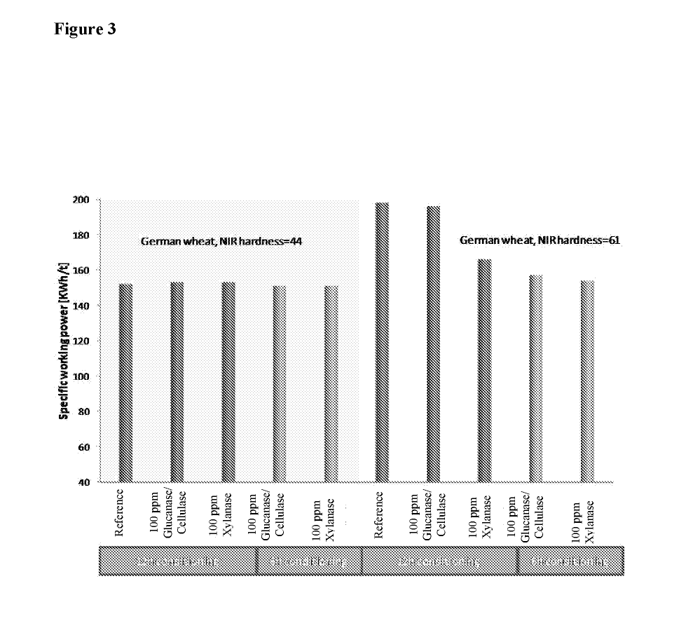

A significant part of product costs in flour milling comes from high energy consumption. An important tool to reduce this energy input is the conditioning of the grain. The two references in FIG. 3 illustrate that energy consumption is related to grain hardness; the softer the grain, the lower energy consumption. Grain is softer after conditioning whereby the energy consumption is reduced. The enzymes compositions reduce the hardness of the grain significantly even with reduced conditioning time. The addition of the enzymes can save approx. 5%-20% energy, e.g. approx. 10%-20% energy, see also Tab. 1. The present inventors have discovered that there is a lower boundary. No significant energy savings can/could be achieved if soft wheat with NIR-hardness lower than 50 NIR was milled without compromising extraction rates and flour quality.

The enzyme compositions reduce the hardness of the grain and with it the resistance of the grain when milling, resulting on less wear of the rolls. Therefore another potential for cost saving is extending maintenance intervals.

Flour Qualities

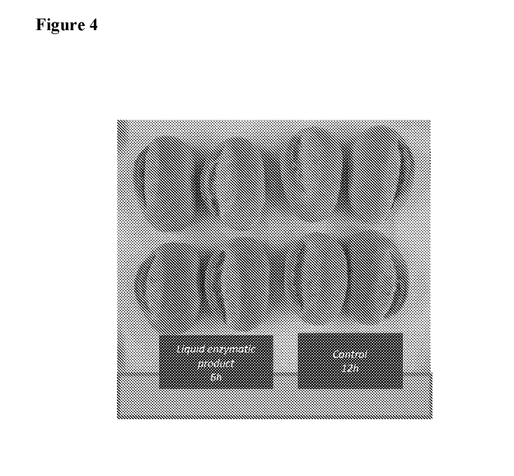

The enzymes compositions do not affect produced flour quality significantly as the added enzymes are located between the outer layers of the bran and the aleurone layer. The enzymes remain in the bran fraction after the sieving steps and are separated from the main flour. No added enzyme activity could/can be measured in the produced bread flour. Several trials with different wheat varieties on different continents confirmed this. Tab. 2 shows an example of a flour analysis from low protein Argentinean wheat with and without the enzymes compositions. All trials indicate that the added enzymes do not change the flour quality significantly. Baking trials shown in FIG. 4 confirm this.

TABLE 2
Flour analysis for low protein Argentinean wheat

| | | | |
|---|---|---|---|
| | | Liquid enzymatic | Liquid enzymatic |
| | Reference | composition | composition |
| The conventional | (12 h) | (12 h) | (6 h) |

Farinograph

| | | | |
|---|---|---|---|
| Water abs [%] | 58.5 | 56.1 | 58.5 |
| Development time | 4.7 | 4.3 | 4.7 |
| [min] | | | |
| Stability | 10.2 | 11.5 | 12.2 |

Extensograph

| | | | |
|---|---|---|---|
| Resistance [UE] | 279 | 282 | 270 |
| Extensibility [mm] | 143 | 115 | 130 |
| R/E | 1.9 | 2.7 | 2.1 |

Physical analysis

| | | | |
|---|---|---|---|
| Moisture [%] | 13.7 | 14.1 | 14 |
| Color minolta [L] | 91.4 | 91.7 | 92 |
| Ash [%] | 0.74 | 0.7 | 0.71 |
| Wet gluten [%] | 24.7 | 24.8 | 23.7 |

Baking trials

| | | | |
|---|---|---|---|
| Spec. volume | 10.8 | 10.7 | 10.8 |
| Dough | Normal | No difference | No difference |
| Cut | Normal | Min. opener | Min. opener |
| Color | Normal | No difference | No difference |

Whole Meal Flour

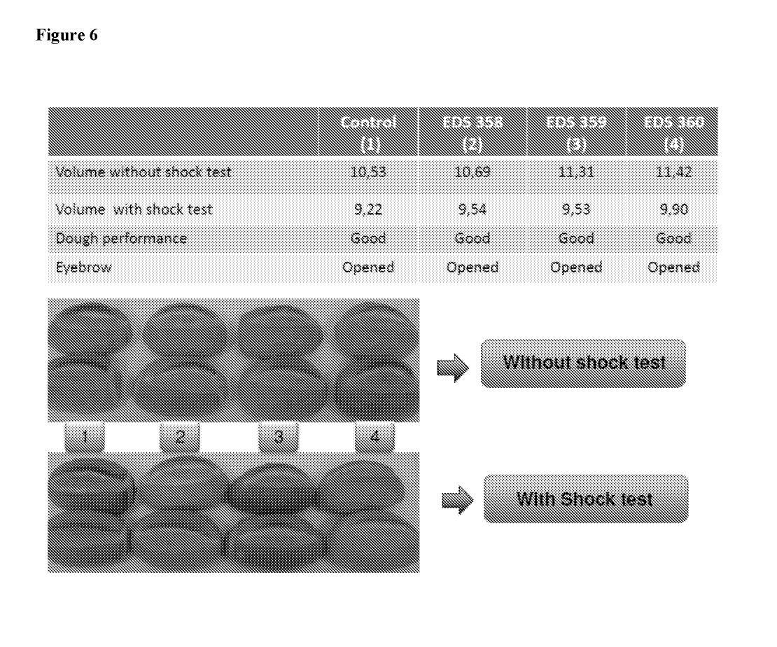

As mentioned before, the enzymes remain in the bran of the grain after milling. FIG. 6 shows the effect of the enzyme on whole meal flour. Whole meal sandwich breads were made with different dosages of the enzymes compositions (EDS 358, EDS 359 and EDS360) and control flour. The columns on the left axis show the spec. volume [ccm/ml] and the line on the right axis demonstrates the dough stickiness after resting time (15 min, 30° C.). FIG. 6 shows a significant increase of the spec. volume. No significant differences in dough stickiness could be seen. However, a higher dose of the cellulase/beta-glucanase enzyme complex caused a slightly more sticky dough (data not shown).

The enzymes compositions are selected xylanases and cellulases. The xylanases are able to split the xylan backbone of the cell walls in the bran while the cellulases open the crystalline structure of the celluloses. Both modifications open the grain structure during conditioning and the water can penetrate much easier into the different layers of the grain. Additionally the unique xylanases support the separation of the endosperm from the aleurone layer. The result is a more sharp separation between the starchy part and the bran and increased extraction rate without a significantly higher ash amount.

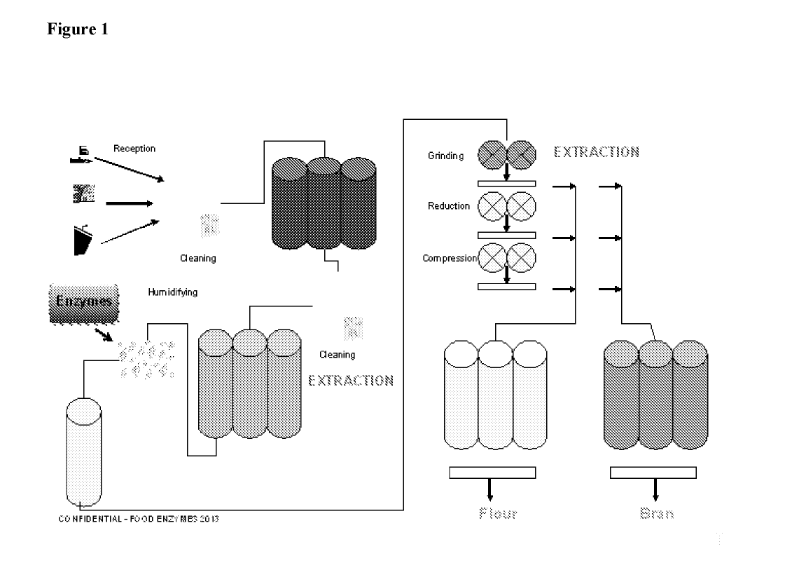

The enzymes compositions are liquid and easy to use. The enzymes can be dosed via a bypass system into the conditioning water. An example can be seen in FIG. 1. This bypass can easily be installed in any existing equipment.

### Example 2

Enzyme Activity Assays:

A. Cell Wall Solubilisation Assay:

In some embodiments, bran solubility can measured using the following assay.

A suspension of wheat bran in (0.1 M)-di-sodium-hydrogen phosphate (0.2 M) buffer, pH 5.0 is prepared to a concentration of 1.33% bran (w/w). From this suspension, aliquots of 750 μl are transferred into Eppendorph tubes under stirring. Each substrate tube is pre-heated for 5 minutes at 40° C. Hereto, 250 μl enzyme solution is added, making the end concentration of substrate 1%. Three dilutions (in duplicate) are made from each enzyme composition according to the invention, with increasing enzyme concentration (e.g. 0.33; 1.0 and 3.0 μg enzyme/gram bran) to each time of determination (0, 30, 60 and 240 minutes). As blank, a heat denatured solution of the enzyme composition is used. The reaction is terminated to the given times, by transferring the tubes to an incubator set at 95° C. Heat denatured samples are kept at 4° C. until all enzyme reactions are terminated. When all enzyme reactions are terminated, Eppendorph tubes are centrifuged to obtain a clear supernatant. The enzymes capability to solubilize bran is expressed as the increase in reducing end groups as determined using PAHBAH (Lever, 1972).

If the bran used contain residual starch, side activities such as amylase activity, may interfere with the above assay, bran solubilisation assay should only be carried out on purified cell wall modifying enzymes (having no amylase activity).

Alternatively the degree of solubilisation may be measured according to the following method:

The degree of solubilisation of a plant material, e.g. cereal bran, can be determined by suspending the insoluble plant material in an extraction buffer (typically 10-25% bran in buffer (w/w)) with and without enzymes, incubate the suspension under stirring and 40 dg C for a controlled time (e.g. 30 to 1440 minutes). After solubilisation, the solubilized material is separated from the insoluble material by centrifugation (20 min, 25000×g, room temp). The dry matter content in the supernatant is determined either by lyophilizing part of the sample, or by a moisture analysis (Moisture analyzer, and ML-50, Buch & Holm, Denmark). All the extraction buffer cannot be recovered using this protocol, however, it is assumed that the concentration of soluble material is the same in the recovered extraction buffer as in the not recovered extraction buffer, why a correction is made for the extraction buffer used in total. Having determined the dry matter content in the soluble fraction, knowing the amount of plant material taking into work and the amount of extraction buffer, the solubilisation degree can be determined using the following equation.

Solubilisation degree=(((gram dry matter/ml supernatant recovered)×(ml extraction buffer used))×100%)/gram plant material taken into work

B. Xylanase Assay (Endo-1,4-Xylanase Activity)

Samples are diluted in citric acid (0.1 M)-di-sodium-hydrogen phosphate (0.2 M) buffer, pH 5.0, to obtain approx. OD590=0.7 in this assay. Three different dilutions of the sample are pre-incubated for 5 minutes at 40° C. At time=5 minutes, 1 Xylazyme tablet (cross-linked, dyed xylan substrate, Megazyme, Bray, Ireland) is added to the enzyme solution in a reaction volume of 1 ml. At time=15 minutes the reaction is terminated by adding 10 ml of 2% TRIS/NaOH, pH 12. Blanks are prepared using 1000 μl buffer instead of enzyme solution. The reaction mixture is centrifuged (1500×g, 10 minutes, 20° C.) and the OD of the supernatant is measured at 590 nm. One xylanase unit (XU) is defined as the xylanase activity increasing OD590 with 0.025 per minute.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Sequences:
AtuXyn3, Aspergillus tubingensis (SEQ ID NO: 1),
302 aa:
QASVSIDTKFKAHGKKYLGNIGDQYTLTKNSKTPAIIKADFGALTPEN
SMKWDATEPSRGQFSFSGSDYLVNFAQSNNKLIRGHTLVWHSQLPSWV
QAITDKNTLIEVMKNHITTVMQHYKGKIYAWDVVNEIFNEDGSLRDSV
FYQVIGEDYVRIAFETARAADPNAKLYINDYNLDSASYPKLTGMVSHV
KKWIEAGIPIDGIGSQTHLSAGGGAGISGALNALAGAGTKEIAVTELD
IAGASSTDYVEVVEACLDQPKCIGITVWGVADPDSWRSSSTPLLFDSN
YNPKPAYTAIANAL
TerXyn1, Geosmithia emersonii (Taleromyces
emersonii) (SEQ ID NO: 2):
AGLNTAAKAIGLKYFGTATDNPELSDTAYETQLNNTQDFGQLTPANSM
KWDATEPEQNVFTFSAGDQIANLAKANGQMLRCHNLVWYNQLPSWVTS
GSWTNETLLAAMKNHITNVVTHYKGQCYAWDVVNEALNDDGTYRSNVE
YQYIGEAYIPIAFATAAAADPNAKLYYNDYNIEYPGAKATAAQNLVKL
VQSYGARIDGVGLQSHFIVGETPSTSSQQQNMAAFTALGVEVAITELD
IRMQLPETEALLTQQATDYQSTVQACANTKGCVGITVWDWTDKYSWVP
STFSGYGDACPWDANYQKKPAYEGILTGLGQTVTSTTYIISPTTSVGT
GTTTSSGGSGGTTGVAQHWEQCGGLGWTGPTVCASGYTCTVINEYYSQ
CL
AtuXyn4, Aspergillus tubingensis (SEQ ID NO: 3):
EPIEPRQASVSIDTKFKAHGKKYLGNIGDQYTLTKNSKTPAIIKADFG
ALTPENSMKWDATEPSRGQFSFSGSDYLVNFAQSNNKLIRGHTLVWHS
QLPSWVQSITDKNTLIEVMKNHITTVMQHYKGKIYAWDVVNEIFNEDG
SLRDSVFYKVIGEDYVRIAFETARAADPNAKLYINDYNLDSASYPKLT
GMVSHVKKWIAAGIPIDGIGSQTHLSAGGGAGISGALNALAGAGTKEI
AVTELDIAGASSTDYVEVVEACLNQPKCIGITVWGVADPDSWRSSSTP
LLFDSNYNPKPAYTAIANAL
AacXyn2, Aspergillus aculeatus (SEQ ID NO: 4):
MVGLLSITAALAATVLPNIVSAVGLDQAAVAKGLQYFGTATDNPELTD
IPYVTQLNNTADFGQITPGNSMKWDATEPSQGTFTFTKGDVIADLAEG
NGQYLRCHTLVWYNQLPSWVTSGTWTNATLTAALKNHITNVVSHYKGK
CLHWDVVNEALNDDGTYRTNIFYTTIGEAYIPIAFAAAAAADPDAKLF
YNDYNLEYGGAKAASARAIVQLVKNAGAKIDGVGLQAHFSVGTVPSTS
SLVSVLQSFTALGVEVAYTEADVRILLPTTATTLAQQSSDFQALVQSC
VQTTGCVGFTIWDWTDKYSWVPSTFSGYGAALPWDENLVKKPAYNGLL
AGMGVTVTTTTTTTTATATGKTTTTTTGATSTGTTAAHWGQCGGLNWS
GPTACATGYTCTYVNDYYSQCL
TreXyn3, Trichoderma reesei (SEQ ID NO: 5):
MKANVILCLLAPLVAALPTETIBLDPELAALRANLTERTADLWDRQAS
QSIDQLIKRKGKLYFGTATDRGLLQREKNAAIIQADLGQVTPENSMKW
QSLENNQGQLNWGDADYLVNFAQQNGKSIRGHTLIWHSQLPAWVNNIN
NADTLRQVIRTHVSTVVGRYKGKIRAWDVVNEIFNEDGTLRSSVFSRL
LGEEFVSIAFRAARDADPSARLYINDYNLDRANYGKVNGLKTYVSKWI
SQGVPIDGIGSQSHLSGGGGSGTLGALQQLATVPVTELAITELDIQGA
PTTDYTQVVQACLSVSKCVGITVWGISDKDSWRASTNPLLFDANFNPK
PAYNSIVGILQ
TreXyn5, Trichoderma reesei (SEQ ID NO: 6):
QCIQPGTGYNNGYFYSYWNDGHGGVTYCNGPGGQFSVNWSNSGNFVGG
KGWQPGTKNRVINFSGSYNPNGNSYLSVYGWSRNPLIEYYIVENFGTY
NPSTGATKLGEVTSDGSVYDIYRTQRVNQPSIIGTATFYQYWSVRRNH
RSSGSVNTANHFNAWAQQGLTLGTMDYQIVAVEGYFSSGSASITVSD
BsuXyn3, Bacillus subtilis xylanase variant
(SEQ ID NO: 7):
ASTDYWQNWTFGGGIVNAVNGSGGNYSVNWSNTGNFVVGKGWTTGSPF
RTINYNAGVWAPNGNGYLTLYGWTRSPLIEYYVVDSWGTYRPTGTYKG
TVKSDGGTYDIYTTTRYNAPSIDGDDTTFTQYWSVRQSKRPTGSNATI
TFSNHVNAWKSHGMNLGSNWAYQVMATEGYQSSGSSNVTVW
BsuXyn4, Bacillus subtilis xylanase variant
(SEQ ID NO: 8):
ASTDYWQNWTDGYGIVNAVNGSGGNYSVNWSNTGNFVVGKGWTTGSPF
RTINYNAGVWAPNGNGYLTLYGWTRSPLIEYYVVDSWGTYRPTGTYKG
TVYSDGGWYDIYTATRDNAPSIDGDFTTFTQYWSVRQSKRPTGSNATI
TFSNHVNAWRSHGMDLGSNWAYQVMATEGYLSSGSSNVTVW

1. A method for conditioning a cereal grain, the method comprising the steps of:
a. providing a cereal grain comprising one or more β-glucans and one or more arabinoxylans;
b. adding water in combination with a liquid composition comprising one or more cell-wall modifying enzyme(s) to the cereal grain; and
c. conditioning the cereal grain for a specific amount of time for the cereal grain to absorb the water in the presence of said one or more cell-wall modifying enzyme(s).

2. The method according to claim 1, wherein said cereal grain is wheat.

3. The method according to claim 2, wherein said one or more cell-wall modifying enzyme is selected from the group consisting of a xylanase, and a cellulase, such as cellobiohydrolases, beta-glucosidases, endo-glucanases, and beta-glucanase.

4. The method according to claim 3, wherein the liquid composition further comprises one or more enzymes selected from the group consisting of arabinofuranosidase, xylosidase, mannanase, alpha-galactosidase, beta-glucuronidase, and beta-galactosidase.

5. The method according to claim 3, wherein the liquid composition further comprises one or more enzymes selected from the group consisting of xylosidase, expansin-like and trypsin-like proteases.

6. The method according to claim 5, wherein said conditioning is performed for more than 6 hours, such as for more than 8, 10, 12, 14, 16, 18, 20, 22, 24, 26, 28, 30, 32, 34, 36, 38, or 40 hours.

7. The method according to claim 6, wherein said conditioning is performed for less than 40 hours, such as for less than 38, 36, 34, 32, 30, 28, 26, 24, 22, 20, 18, 16, 14, 12, 10, or 8 hours.

8. The method according to claim 7, wherein said composition comprising one or more cell-wall modifying enzyme(s) is a liquid, such as an aqua formulation.

9. The method according to claim 8, wherein said composition comprising one or more cell-wall modifying enzyme(s) is an aqua formulation comprising the enzymes secreted from the fermentation of the genus Trichoderma, such as Trichoderma reesei, such as beta-glucanases or cellulases.

10. The method of claim 9 wherein said composition comprises one or more beta-glucanases and/or cellulases.

11. The method of claim 10, wherein said composition further comprises one or more enzymes exhibiting xylanase activity.

12. The method according to claim 11, wherein said composition further comprises one or more enzymes exhibiting beta-xylosidase activity.

13. The method according to claim 12, wherein said composition further comprises one or more enzymes exhibiting mannanase activity.

14. The method according to claim 13, wherein said composition further comprises one or more enzymes exhibiting arabinofuranosidase activity.

15. The method according to claim 14, wherein said composition further comprises one or more enzymes exhibiting alpha-galactosidase activity

16. The method according to claim 15, wherein said composition further comprises one or more enzymes exhibiting beta-glucuronidase activity.

17. The method according to claim 16, wherein said composition further comprises one or more enzymes exhibiting beta-galactosidase activity.

18. The method of claim 9, wherein said composition comprises one or more enzymes having an amino acid sequence having at least 80% identity to the enzymes selected from the group of enzymes having GenBank accession no. consisting of M16190, M15665, M19373, AB003694, Y11113, Z33381, AY281371, AY281372, AY281373, U09580, AB003110, AY281374, AY281375, AY281377, AY281378, AY281379, X69574, X69573, AB036796, Z69257, Z69256, AY281376, Z69252, AY281369, L25310, Z69253, Z69254, Z69255, Z68706, AJ549427, AJ245918, AY281370, AY281368, or any functional fragment thereof.

19. The method of claim 9, wherein said composition comprises one or more enzymes having at least 80% identity to the enzymes selected from the group of enzymes having locus no. (from genome.jgi-psf.org/Trire2/Trire2.home.html) of ORF_123283, ORF_76210, ORF_55319, ORF_54219, ORF_123989, ORF_123989, ORF_123989, ORF_123989, ORF_72567, ORF_72567, ORF_72567, ORF_72567, ORF_72567, ORF_72567, ORF_72567, ORF_72567, ORF_72567, ORF_72567, ORF_122081, ORF_120312, ORF_120312, ORF_123232, ORF_123232, ORF_49081, ORF_49081, ORF_49081, ORF_49081, ORF_27554, ORF_121127, ORF_121127, ORF_74223, ORF_123818, ORF_111849, ORF_56996, ORF_76672, and ORF_73897.

20. The method of claim 19 wherein said composition comprises two or more independently selected enzymes exhibiting beta-glucanase activity and at least one enzyme exhibiting xylanase activity.

21. The method of claim 20 wherein the one or more enzyme has at least 81, 82, 83, 84, 85, 86, 87, 88, 89, 90, 91, 92, 93, 94, 95, 96, 97, 98 or 99% identity with any one amino acid sequence.

22. The method according to claim 21, wherein said beta-glucanase and said cellulase is present in an amount of 10,000 to 1,000,000 AZO BBG U per ton cereal grain (75,000-340,000), and 100,000 to 10×10⁶ IU per ton cereal grain (310,000-1,516,000) respectively.

23. The method according to claim 22, wherein said composition comprising one or more cell-wall modifying enzyme(s) is an aqua formulation comprising the enzymes secreted from the fermentation of the genus Bacillus, such as Bacillus subtilis, such as a bacterial xylanase.

24. The method according to claim 22, wherein said composition comprises one or more cell-wall modifying enzyme(s) is an aqua formulation comprising an enzyme having xylanase activity, which enzyme comprises an amino acid sequence having at least 80% identity with any one of the amino acid sequences selected from SEQ ID NO:1-SEQ ID NO:8, or any functional fragment thereof.

25. In The method according claim 24, wherein said composition comprises one or more enzymes comprising an amino acid sequence having at least 80% identity with any one of the amino acid sequences elected from SEQ ID NO:1; SEQ ID NO:2, SEQ ID NO:7 and SEQ ID NO:8, or any functional fragment thereof

26. The method according to claim 22, further comprising one or more beta-glucanase.

27. The method according to claim 26, wherein said xylanase is present in an amount of 1×10⁶ to 100×10⁶ units per ton cereal grain (9×10⁶ to 52×10⁶).

28. The method according to claim 27, wherein said composition in step a further comprises one or more oxidase.

29. The method of claim 28 wherein, said adding water comprises spraying the cereal and wherein said one or more cell-wall modifying enzyme are added one or more times, or constantly, during said spraying.

30. The method of claim 29, wherein the cereal reaches a moisture content of between about 12 to about 17%, and wherein said moisture content is reached in 12 hours or less.

31. The method of claim 30, wherein:
(i) said cereal has 0.5-10% W/W of β-glucans; or
(ii) said cereal has 1-10% W/W of arabinoxylans; or
(iii) the amount of high molecular weight β-glucans in the cereal is decreased at least 50%; or
(iv) the amount of high molecular weight β-glucans in the cereal is decreased at least 80%; or
(v) the amount of high molecular weight arabinoxylans in the cereal is decreased at least 50%; or
(vi) wherein the cereal is a hard cereal grain, and said liquid composition comprising one or more cell-wall modifying enzymes is added during the conditioning process at a concentration of 50-200 ppm for about 6 to 12 hours; or
(vii) wherein the cereal is a mid-hard cereal grain, and said liquid composition comprising one or more cell-wall modifying enzymes is added during the conditioning process at a concentration of 50-200 ppm for about 6 to 12; or
(viii) wherein the cereal is a soft cereal grain, and said liquid composition comprising one or more cell-wall modifying enzymes is added during the conditioning process at a concentration of 50-150 ppm for about 6 to 12; or
(ix) wherein the cereal is a soft cereal grain, and said liquid composition comprising one or more cell-wall modifying enzymes is added during the conditioning process at a concentration of 50-150 ppm for about 6 or less.

32. The method of claim 31, wherein the amount of high molecular weight β-glucans in said cereal is 150 mg/l or less, but optionally no less than 50 mg/l, and the amount of arabinoxylans in said conditioned cereal is at 2000 mg/l or less, but optionally no less than 1000 mg/l.

33. The method of claim 32, said method comprises spraying water to said cereal, where said one or more cell wall modifying enzymes are added one or more times during said spraying, and wherein the amount of high molecular weight β-glucans in said conditioned cereal is 150 mg/l or less, but optionally no less than 50 mg/l, and the amount of arabinoxylans in said cereal is at 2000 mg/l or less, but optionally no less than 1000 mg/l.

34. The method of claim 34, wherein the wet gluten in said conditioned cereal is at least 24%, 25%, 27%, 29%, 30%, 31% or 32%.

35. A method for the extraction of flour from a cereal grain, the method comprising the steps of:
a) conditioning the cereal grain in a method according to claim 1; and
b) milling the cereal grain and separate the flour from the bran of the cereal grain.

36. The method of claim 35, wherein the extraction rate increases at least about 0.5%, such as from at least about 0.6, 0.7, 0.8, 0.9, 1.0, 1.1, 1.2, 1.3, 1.4, 1.5, 1.6, 1.7, 1.8, 1.9, or 2% compared to a negative control cereal conditioned without said enzyme composition.

37. System suitable for operating a method according to claim 19, wherein water containing one or more cell-wall modifying enzyme is sprayed to a composition of cereal grains during the conditioning of said cereal grains, said system containing a dosing system to adjust the amount of said enzyme being added to said water containing one or more cell-wall modifying enzyme.

38. The system according to claim 37 further comprising a mixing mechanism.

39. An aqua composition comprising an expression product obtained by fermentation of a species of the genus Trichoderma; which expression product comprises a beta-glucanase (EC 3.2.1.6) and a cellulase (EC 3.2.1.4), wherein said beta-glucanase is present in an amount of 1000-2000 AZO BBG U per gram aqua composition and said cellulase is present in an amount of 6000-8000 IU per gram aqua composition.

40. The aqua composition according to claim 39, wherein said expression product obtained by fermentation of the genus Trichoderma is from the species Trichoderma reesei.

41. The aqua composition of claim 39, wherein said composition comprises one or more enzymes having an amino acid sequence having at least 80% identity to the enzymes selected from the group of enzymes having GenBank accession no. consisting of M16190, M15665, M19373, AB003694, Y11113, Z33381, AY281371, AY281372, AY281373, U09580, AB003110, AY281374, AY281375, AY281377, AY281378, AY281379, X69574, X69573, AB036796, Z69257, Z69256, AY281376, Z69252, AY281369, L25310, Z69253, Z69254, Z69255, Z68706, AJ549427, AJ245918, AY281370, AY281368, or any functional fragment thereof.

42. The aqua composition of claim 39, wherein said composition comprises one or more enzymes having at least 80% identity to the enzymes selected from the group of enzymes having locus no. (from genome.jgi-psf.org/Trire2/Trire2.home.html) of ORF_123283, ORF_76210, ORF_55319, ORF_54219, ORF_123989, ORF_123989, ORF_123989, ORF_123989, ORF_72567, ORF_72567, ORF_72567, ORF_72567, ORF_72567, ORF_72567, ORF_72567, ORF_72567, ORF_72567, ORF_72567, ORF_122081, ORF_120312, ORF_120312, ORF_123232, ORF_123232, ORF_49081, ORF_49081, ORF_49081, ORF_49081, ORF_27554, ORF_121127, ORF_121127, ORF_74223 ORF_123818 ORF_111849 ORF_56996, ORF_74223, ORF_123818, ORF_111849, ORF_56996, 76672, and ORF_73897.

43. The method of claim 42 wherein said composition comprises two or more independently selected enzymes exhibiting beta-glucanase activity and at least one enzyme exhibiting xylanase activity.

44. An aqua composition comprising a xylanase (EC 3.2.1.8), wherein said xylanase is present in an amount of 100000-300000 units per gram aqua composition.

45. The aqua composition according to claim 44, comprising an expression product obtained by fermentation of a species of the genus Bacillus, such as a species Bacillus subtilis.

46. The aqua composition according to claim 44, wherein said xylanase comprises an amino acid sequence having at least 80% identity with any one of the amino acid sequences selected from SEQ ID NO:1-SEQ ID NO:8, or any functional fragment thereof.

47. The aqua composition according to claim 46, wherein said composition comprises one enzymes comprising an amino acid sequence having at least 80% identity with any one of the amino acid sequences elected from SEQ ID NO:1; SEQ ID NO:2, SEQ ID NO:7 and SEQ ID NO:8, or any functional fragment thereof

48. The aqua composition according to claim 47, further comprising one or more beta-glucanase.

49. Use of a system according to claim 37, or an aqua composition according to claim 48 in a process of cereal grain conditioning.

50. Flour or cereal bran obtained from a method according to claim 35 or any food product obtained therefrom, such as a bread product.


SEQUENCE LISTING (8 sequences)

SEQ ID NO: 1 - protein, 302 aa, Aspergillus tubingensis
QASVSIDTKFKAHGKKYLGNIGDQYTLTKNSKTPAIIKADFGALTPENSMKWDATEPSRGQFSFSGSDYLVNFAQSNNKLIRGHTLVWHSQLPSWVQAITDKNTLIEVMKNHITTVMQHYKGKIYAWDVVNEIFNEDGSLRDSVFYQVIGEDYVRIAFETARAADPNAKLYINDYNLDSASYPKLTGMVSHVKKWIEAGIPIDGIGSQTHLSAGGGAGISGALNALAGAGTKEIAVTELDIAGASSTDYVEVVEACLDQPKCIGITVWGVADPDSWRSSSTPLLFDSNYNPKPAYTAIANAL

SEQ ID NO: 2 - protein, 386 aa, Talaromyces emersonii
AGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 3 - protein, 308 aa, Aspergillus tubingensis
EPIEPRQASVSIDTKFKAHGKKYLGNIGDQYTLTKNSKTPAIIKADFGALTPENSMKWDATEPSRGQFSFSGSDYLVNFAQSNNKLIRGHTLVWHSQLPSWVQSITDKNTLIEVMKNHITTVMQHYKGKIYAWDVVNEIFNEDGSLRDSVFYKVIGEDYVRIAFETARAADPNAKLYINDYNLDSASYPKLTGMVSHVKKWIAAGIPIDGIGSQTHLSAGGGAGISGALNALAGAGTKEIAVTELDIAGASSTDYVEVVEACLNQPKCIGITVWGVADPDSWRSSSTPLLFDSNYNPKPAYTAIANAL

SEQ ID NO: 4 - protein, 406 aa, Aspergillus aculeatus
MVGLLSITAALAATVLPNIVSAVGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 5 - protein, 347 aa, Trichoderma reesei
MKANVILCLLAPLVAALPTETIHLDPELAALRANLTERTADLWDRQASQSIDQLIKRKGKLYFGTATDRGLLQREKNAAIIQADLGQVTPENSMKWQSLENNQGQLNWGDADYLVNFAQQNGKSIRGHTLIWHSQLPAWVNNINNADTLRQVIRTHVSTVVGRYKGKIRAWDVVNEIFNEDGTLRSSVFSRLLGEEFVSIAFRAARDADPSARLYINDYNLDRANYGKVNGLKTYVSKWISQGVPIDGIGSQSHLSGGGGSGTLGALQQLATVPVTELAITELDIQGAPTTDYTQVVQACLSVSKCVGITVWGISDKDSWRASTNPLLFDANFNPKPAYNSIVGILQ

SEQ ID NO: 6 - protein, 191 aa, Trichoderma reesei
QCIQPGTGYNNGYFYSYWNDGHGGVTYCNGPGGQFSVNWSNSGNFVGGKGWQPGTKNRVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVSD

SEQ ID NO: 7 - protein, 185 aa, Bacillus subtilis
ASTDYWQNWTFGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDDTTFTQYWSVRQSKRPTGSNATITFSNHVNAWKSHGMNLGSNWAYQVMATEGYQSSGSSNVTVW

SEQ ID NO: 8 - protein, 185 aa, Bacillus subtilis
ASTDYWQNWTDGYGIVNAVNGSGGNYSVNWSNTGNFVVGKGWTTGSPFRTINYNAGVWAPNGNGYLTLYGWTRSPLIEYYVVDSWGTYRPTGTYKGTVYSDGGWYDIYTATRDNAPSIDGDFTTFTQYWSVRQSKRPTGSNATITFSNHVNAWRSHGMDLGSNWAYQVMATEGYLSSGSSNVTVW